(19) United States
(12) Patent Application Publication
(10) Pub. No.: US 2016/0136346 A1
(43) Pub. Date: May 19, 2016

(54) HEMODIALYSIS APPARATUS

(71) Applicant: Taebeom Cho, Daejeon (KR)

(72) Inventor: Taebeom Cho, Daejeon (KR)

(21) Appl. No.: 14/944,543

(22) Filed: Nov. 18, 2015

(30) Foreign Application Priority Data
Nov. 18, 2014 (KR) 10-2014-0160448
Nov. 18, 2014 (KR) 10-2014-0160449
Nov. 25, 2014 (KR) 10-2014-0164875

(51) Int. Cl. A61M 1/16; A61M 39/24

(52) U.S. Cl. CPC A61M 1/1601; A61M 1/1654; A61M 39/24

(57) ABSTRACT

Provided is a hemodialysis apparatus including a hemodialyzer in which mass transfer occurs between blood and dialysate, a blood tube connecting the hemodialyzer and a patient to allow blood to flow therethrough, a blood flow controller which controls the blood flow passage through the blood tube, a dialysate supply pump supplying dialysate to the hemodialyzer, and a dialysate discharge pump discharging dialysate having passed through the hemodialyzer. The dialysate supply pump and the dialysate discharge pump further include a tube pressurizing member and a one-way valve or a roller and a roller driver. The blood tube includes a first blood tube in which blood of a patient is supplied to the hemodialyzer and a second blood tube in which blood passing through the hemodialyzer is returned to the patient.

### CROSS-REFERENCE TO RELATED APPLICATIONS

Pursuant to 35 U.S.C. §119, this application claims the benefit of priority to Korean Patent Application No. 10-2014-0160448 filed Nov. 18, 2014, Korean Patent Application No. 10-2014-0160449 filed Nov. 18, 2014, and Korean Patent Application No. 10-2014-0164875 filed Nov. 25, 2014 in the Korean Intellectual Property Office, the entire contents of which are hereby incorporated by reference in their entirety.

### BACKGROUND

The present invention relates to a hemodialysis apparatus configured to improve water exchange and mass transfer between blood and dialysate by quickly changing the dialysate pressure in a hemodialyzer using a pulsatile dialysate flow.

When there is a kidney dysfunction, water and waste products that have to be discharged out of body accumulate in blood and imbalance of electrolytes in the body occurs. Most commonly performed to improve such a kidney failure symptom, is hemodialysis which is to circulate blood out of body and rid the blood of the accumulated uremic toxin and excess water by a semi-permeable dialysis membrane. Hemodialysis is a method of seeking an electrolyte balance and ridding the body fluid of uremic toxin and excess water, taking advantages of diffusion applied due to the concentration difference and filtration applied due to the pressure difference between blood and dialysate.

Most commonly used of hemodialyzer is the type that is a cylinder-shape container charged with a bundle of hollow fiber membranes and port-processed at both ends thereof by use of a synthetic resin like polyurethane. It is because the hollow fiber hemodialyzer has excellent mass-transfer efficiency resulting from large effective surface area between blood and dialysate compared to the small size as a whole.

A hemodialysis apparatus includes a hemodialyzer in which mass transfer occurs between blood and dialysate, a blood pump designed to circulate a patient's blood, a dialysate pump that supplies or discharges dialysate. Blood and dialysate each decrease their hydraulic pressure while passing through a hemodialyzer. Since blood and dialysate flow in opposite directions inside the hemodialyzer, a filtration occurs at the proximal part of the hemodialyzer such that water in the blood moves toward dialysate compartment because blood pressure is higher than dialysate pressure, while a backfiltration occurs at the distal part such that water in the dialysate moves toward blood domain for the same reason.

When a filtration takes place, wastes in blood are also eliminated, which is referred to as a convective mass transfer. It is known that uremic toxins of medium molecular size are efficiently removed by the convective mass transfer and thus dialysis efficiency and prognosis on patients have greatly improved. However, there is a big hurdle in the effort to improve dialysis efficiency by the convective mass transfer, because hemodialyzers in typical hemodialysis apparatuses are limited in size and blood flow rate is restrictively allowed to be increased in consideration of the weight and blood vessel condition of a patient.

### SUMMARY OF THE DISCLOSURE

The present invention provides a hemodialysis apparatus, which improves the hemodialysis efficiency by allowing a pressure difference between blood and dialysate to be alternately changed into positive and negative values and thereby maximizing water exchange between blood and dialysate, and enables simplification and miniaturization of the hemodialysis apparatus by eliminating the use of a blood pump.

Embodiments of the present invention provide hemodialysis apparatuses including: a hemodialyzer in which mass transfer occurs between blood and dialysate, a first blood tube connecting a patient and the hemodialyzer to allow blood of a patient to be supplied to the hemodialyzer, a second blood tube connecting the hemodialyzer and a patient to allow blood having passed through the hemodialyzer to be returned to a patient, a blood flow controller controlling a blood flow passage through the first and second blood tubes, a dialysate supply pump supplying dialysate to the hemodialyzer, and a dialysate discharge pump discharging dialysate having passed the hemodialyzer.

A dialysate tube in which dialysate flows includes a dialysate supply tube and a dialysate discharge tube. Also, the dialysate supply tube further includes a first dialysate supply tube and a second dialysate supply tube, and the dialysate discharge tube includes a first dialysate discharge tube and a second dialysate discharge tube. Dialysate can be stored in a dialysate supply tank and then supplied to the hemodialyzer, and used dialysate having passed the hemodialyzer may be collected in a dialysate recovery tank.

The hemodialyzer includes a hemodialyzer container having an internal space and a hemodialysis membrane accommodated in the internal space of the hemodialyzer container. The hemodialyzer container includes a blood inlet disposed at one end thereof and a blood outlet disposed at the other end thereof. Also, a dialysate inlet and a dialysate outlet may be provided on the outer surface of the hemodialyzer container.

The dialysate supply pump and the dialysate discharge pump may include a tube pressurizing member which pressurizes the dialysate tube to transfer dialysate therein, a supporting wall which supports the dialysate tube, and a tube one-way valve provided at both sides of the dialysate tube pressurized by the tube pressurizing member to prevent dialysate in the tube from flowing backward.

In this instance, dialysate flow passage through the dialysate tube may be blocked when the dialysate tube is pressurized by the tube pressurizing member. For this, the tube pressurizing member may be provided with a tube pressurizing member protrusion on the pressurizing surface of the tube pressurizing member. Alternatively, the supporting wall may be provided with a supporting wall protrusion on the supporting wall.

The dialysate supply pump and the dialysate discharge pump may be modified to have a structure including a roller squeezing the dialysate tube to transfer dialysate therein and a roller driver to operate the roller. When dialysate is transferred by the rotational movement of the roller, the roller driver may use various structures to rotate the roller.

The blood flow controller which controls blood flow passage through the blood tubes may include a housing having an internal space, a flow port disposed on the housing, a rotor disposed inside the housing to connect flow passage between the flow ports, and a rotor driver for rotating the rotor. In addition, the blood flow controller may be modified into a structure includes a flow-blocking member reciprocating in a straight line to compress the blood tube, a flow-blocking member driver providing a straight force to the flow-blocking member, and a flow-blocking wall supporting the blood tube compressed by the flow-blocking member. Another example of the blood flow controller may include a blood tube one-way valve disposed in the blood flow tube to ensure blood to flow in a pre-determined direction. In addition, the hemodialysis apparatus may further include a volume chamber to store dialysate

The flow of dialysate in the hemodialysis apparatus may include: a first phase where the dialysate supply tube is pressurized by the supply tube pressurizing member and the dialysate therein is supplied to the hemodialyzer, while the dialysate discharge tube is pressurized and blocked by the discharge tube pressurizing member; a second phase where the dialysate discharge tube is expanded by the discharge tube pressurizing member and the dialysate of the hemodialyzer is transferred to the dialysate discharge tube expanded, while the dialysate supply tube is pressurized and blocked by the supply tube pressurizing member; and a third phase where the dialysate supply tube is filled with fresh dialysate due to the expansion of the dialysate supply tube by the supply tube pressurizing member and the dialysate of the dialysate discharge tube is discarded therefrom due to the compression of the dialysate discharge tube by the discharge tube pressurizing member.

During the first phase, the pressure of the dialysate flow region inside the hemodialyzer increases and backfiltration in which water in dialysate moves toward the blood flow region occurs. Blood of the hemodialyzer is then returned to a patient due to the backfiltration. In contrast, during the second phase, the pressure of the dialysate flow region inside the hemodialyzer decreases and filtration occurs. Thus, blood of a patient is supplied to the hemodialyzer through the first blood tube due to the filtration. That is, a cycle of the expansion and compression of the dialysate supply pump and the dialysate discharge pump configures a cycle of filtration and backfiltration inside the hemodialyzer and simultaneously enables blood of a patient to be supplied to the hemodialyzer and then returned. Water and waste products are removed during the filtration and lost water is supplemented during the backfiltration.

A hemodialysis apparatus according to a second embodiment of the present invention includes the hemodialyzer, the first blood tube, the second blood tube, a flow control device controlling blood and dialysate flow passage through the blood tube and the dialysate tube, a dialysate supply pump supplying dialysate to the hemodialyzer, and a dialysate discharge pump discharging dialysate having passed the hemodialyzer.

The dialysate supply pump and the dialysate discharge pump include a cylinder having an internal space, a piston disposed inside the cylinder, and a piston driver allowing the piston to reciprocate. The piston driver includes various structures that can compress or expand the supply pump cylinder and the discharge pump cylinder by pushing or pulling the piston. The supply pump cylinder and the discharge pump cylinder are simultaneously compressed or expanded.

The flow control device controls the flow passage through the blood tube, the dialysate supply tube, and the dialysate discharge tube. In particular, the flow control device may alternately block the first blood tube, the first dialysate supply tube, and the first dialysate discharge tube, or the second blood tube, the second dialysate supply tube, and the second dialysate discharge tube. The flow control device may comprise a housing, a flow port disposed on the housing, a rotor disposed inside the housing to connect flow passage between the flow ports, and a rotor driver rotating the rotor. Alternatively, the flow control device may have a structure that includes a flow-blocking member to compress the blood and dialysate tubes, a flow-blocking member driver providing a straight force to the flow-blocking member, and a flow-blocking wall supporting the blood and dialysate tubes compressed by the flow-blocking member.

In the same manner, the hemodialysis apparatus according to a second embodiment of the present invention may also have the volume chamber to store dialysate.

The hemodialysis apparatus repeats an expansion phase in which the cylinders are expanded by the pulling of the piston and a compression phase in which the cylinders are compressed. When the cylinders are expanded, the flow control device opens the first blood tube, the first dialysate supply tube and the first dialysate discharge tube, and blocks the second blood tube, the second dialysate supply tube and the second dialysate discharge tube. Due to the expansion of the supply pump cylinder, dialysate flows into the cylinder. Similarly, due to the expansion of the discharge pump cylinder, dialysate of the hemodialyzer flows into the discharge pump cylinder. Simultaneously, since the second dialysate supply tube is blocked, the hydrostatic pressure of the dialysate flow region inside the hemodialyzer is lowered and a filtration in which water and uremic toxin in blood move to the dialysate flow region occurs. The filtration enables blood of a patient to be supplied to the hemodialyzer through the first blood tube that is opened. In addition, dialysate may be stored in the volume chamber during this phase.

On the other hand, when the cylinders are compressed, the flow control device blocks the first blood tube, the first dialysate supply tube and the first dialysate discharge tube, and opens the second blood tube, the second dialysate supply tube and the second dialysate discharge tube. Due to the compression of the discharge pump cylinder, dialysate of the discharge pump cylinder is discharged out of the discharge pump cylinder. Similarly, when the supply pump cylinder is compressed, dialysate inside the cylinder is supplied to the hemodialyzer. At this time, since the first dialysate discharge tube is blocked, the hydrostatic pressure of the dialysate flow region inside the hemodialyzer increases and a backfiltration occurs. Backfiltration enables blood of the hemodialyzer to be returned to a patient through the second blood tube that is opened. Dialysate may be discharged out of the volume chamber during this compression phase.

That is, the compression and expansion of the dialysate supply pump and the dialysate discharge pump configures a cycle of filtration and backfiltration. In the hemodialysis using the hemodialysis apparatus according to embodiments of the present invention, the cycle of filtration and backfiltration is continuously repeated, removing water and waste products during the filtration and supplementing lost water during the backfiltration.

Dialysate pressure increases during the first phase, the supply phase or the compression phase, whereas it decreases during the second phase, the discharge phase, or the expansion phase. When the dialysate pressure fluctuates, the hemodialysis apparatus according to embodiments of the present invention may further include a pressure-relief bypass which connects between the first and second dialysate discharge tube. When the dialysate pressure of the hemodialyzer increases above the permissible range, dialysate of the hemodialyzer may be removed to the second dialysate discharge tube through the pressure-relief bypass. On the contrary, when the dialysate pressure inside the hemodialyzer decreases below the permissible range, dialysate may be supplemented to the hemodialyzer through the pressure-relief bypass so as to compensate the pressure decrease inside the hemodialyzer. The pressure-relief bypass is not limited to be opened or closed by the dialysate pressure of the hemodialyzer. Rather, the pressure-relief bypass can be opened or closed by the pressure of the second dialysate supply tube, the pressure difference of the both tubes connected by the pressure-relief bypass, or the transmembrane pressure (TMP) of the hemodialyzer. The pressure values that can open or close the pressure-relief bypass may be dependent on the hemodialysis membrane that is used.

In addition, the hemodialysis apparatus according to embodiments of the present invention may be further provided with a method to measure the amount of dialysate supplied to the hemodialyzer and the amount of dialysate collected from the hemodialyzer. For example, a balance may be provided to measure the amount of dialysate supplied from the dialysate supply tank and then collected in the dialysate recovery tank. Since water is accumulated in the body of a patient with renal disease due to the absence of the kidney function, it is important to remove excess water from the body as well as remove waste product from the body upon hemodialysis.

Finally, the hemodialysis apparatus may additionally have an auxiliary discharge tube connecting between the first and second dialysate discharge tube, and an auxiliary discharge pump disposed on the auxiliary discharge tube to additionally pull dialysate of the hemodialyzer toward the dialysate recovery tank. In a situation where the supply amount of dialysate by the dialysate supply pump and the discharge amount of dialysate by the dialysate discharge pump are equal to each other, when the auxiliary discharge pump operates, water may be additionally discharged out of blood, thereby removing excess water accumulated in the body of a patient.

### BRIEF DESCRIPTION OF THE DRAWINGS

The accompanying drawings are included to provide a further understanding of the present invention, and are incorporated in and constitute a part of this specification. The drawings illustrate exemplary embodiments of the present invention and, together with the description, serve to explain principles of the present invention. In the drawings:

FIG. 1 is a view illustrating a hemodialysis apparatus according to a first embodiment of the present invention;

FIG. 2 is a cross-sectional view of a hemodialyzer;

FIG. 3 is a view illustrating a hemodialysis apparatus having a dialysate supply pump and a dialysate discharge pump according to a first embodiment of the present invention;

FIG. 4 is an enlarged perspective view of a dialysate supply pump and a dialysate discharge pump including a tube pressurizing member, a supporting wall, and a tube one-way valve;

FIG. 5 is a cross-sectional view illustrating a dialysate supply pump and a dialysate discharge pump having a tube pressurizing member protrusion;

FIG. 6 is a cross-sectional view illustrating a dialysate supply pump and a dialysate discharge pump having a wall protrusion;

FIG. 7 is a view illustrating a hemodialysis apparatus having a roller and a roller driver for a dialysate supply pump and a dialysate discharge pump;

FIG. 8 is a cross-sectional view illustrating a dialysate pump including a roller;

FIGS. 9 to 11 are views illustrating a blood flow controller;

FIGS. 12 and 13 are views illustrating a hemodialysis apparatus having a volume chamber;

FIGS. 14 to 18 are views illustrating an operation of a hemodialysis apparatus according to a first embodiment of the present invention;

FIG. 19 is a view illustrating a hemodialysis apparatus according to a second embodiment of the present invention;

FIGS. 20 and 21 are views illustrating a hemodialysis apparatus having a dialysate supply pump and a dialysate discharge pump according to a second embodiment of the present invention;

FIGS. 22 and 23 are views of a flow control device;

FIGS. 24 and 25 are views illustrating a hemodialysis apparatus having a volume chamber according to a second embodiment of the present invention;

FIGS. 26 and 27 are views illustrating an operation of a hemodialysis apparatus according to a second embodiment of the present invention;

FIGS. 28 to 31 are views illustrating a hemodialysis apparatus having a pressure-relief bypass;

FIG. 32 is an exemplary view of a pressure-relief bypass;

FIGS. 33 to 36 are views illustrating a hemodialysis apparatus having a balance, an auxiliary discharge tube, and an auxiliary discharge pump disposed on the auxiliary discharge tube.

### DETAILED DESCRIPTION OF PREFERRED EMBODIMENTS

Preferred embodiments of the present invention will be described below in more detail with reference to the accompanying drawings. The present invention may, however, be embodied in different forms and should not be constructed as limited to the embodiments set forth herein. Rather, these embodiments are provided so that this disclosure will be thorough and complete, and will fully convey the scope of the present invention to those skilled in the art.

Hereinafter, a hemodialysis apparatus according to a first embodiment of the present invention will be described in detail with reference to the accompanying drawings.

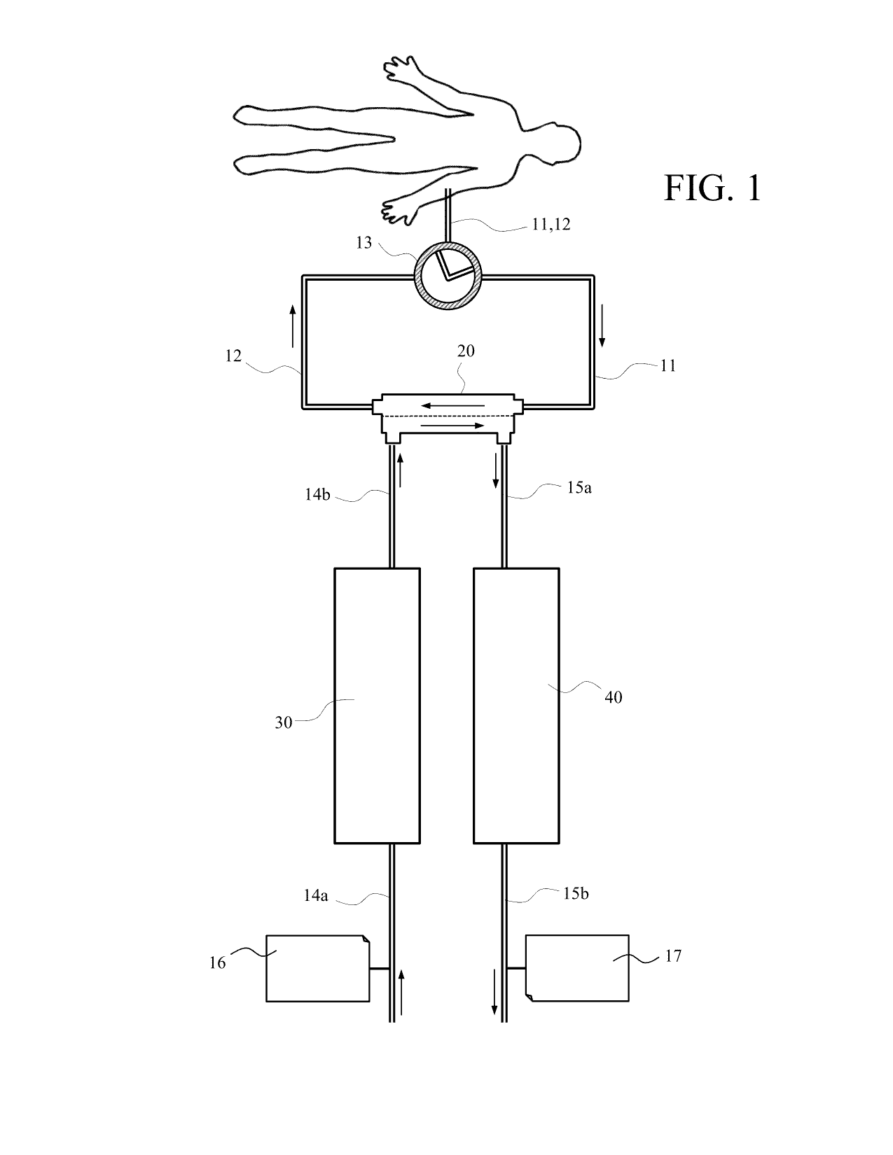

As shown in FIG. 1, a hemodialysis apparatus 10 includes a hemodialyzer 20 in which mass transfer occurs between blood and dialysate, a first blood tube 11 connecting a patient and the hemodialyzer 20 and allowing blood of a patient to be supplied to the hemodialyzer, a second blood tube 12 connecting the hemodialyzer 20 and a patient and allowing blood having passed through the hemodialyzer to be returned to a patient, a blood flow controller 13 controlling a blood flow passage through the first and second blood tubes 11 and 12, a dialysate supply pump 30 supplying dialysate to the hemodialyzer 20, and a dialysate discharge pump 40 discharging dialysate having passed the hemodialyzer.

A dialysate tube in which dialysate flows includes a dialysate supply tube 14 through which dialysate is supplied to the hemodialyzer 20 and a dialysate discharge tube 15 through which dialysate is discharged from the hemodialyzer. Also, the dialysate supply tube 14 further includes a first dialysate supply tube 14a and a second dialysate supply tube 14b through which dialysate is supplied to the dialysate supply pump 30 and the hemodialyzer 20, respectively. Similarly, the dialysate discharge tube 15 includes a first dialysate discharge tube 15a connecting the hemodialyzer 20 and the dialysate discharge pump 40 to allow dialysate of the hemodialyzer to be discharged to the dialysate discharge pump and a second dialysate discharge tube 15b through which dialysate of the dialysate discharge pump is discarded therefrom.

Dialysate may be manufactured by adjusting pH and electrolyte concentration in the ultrapure water prepared through a water treatment system. The fresh dialysate can be stored in a dialysate supply tank 16 and then supplied to the hemodialyzer 20, and used dialysate having passed the hemodialyzer may be collected in a dialysate recovery tank 17. However, dialysate can be supplied directly to the hemodialyzer 20 without being stored in the dialysate supply tank 16 and used dialysate can be discarded without being collected in the dialysate recover tank 17 to inhibit a contamination of dialysate.

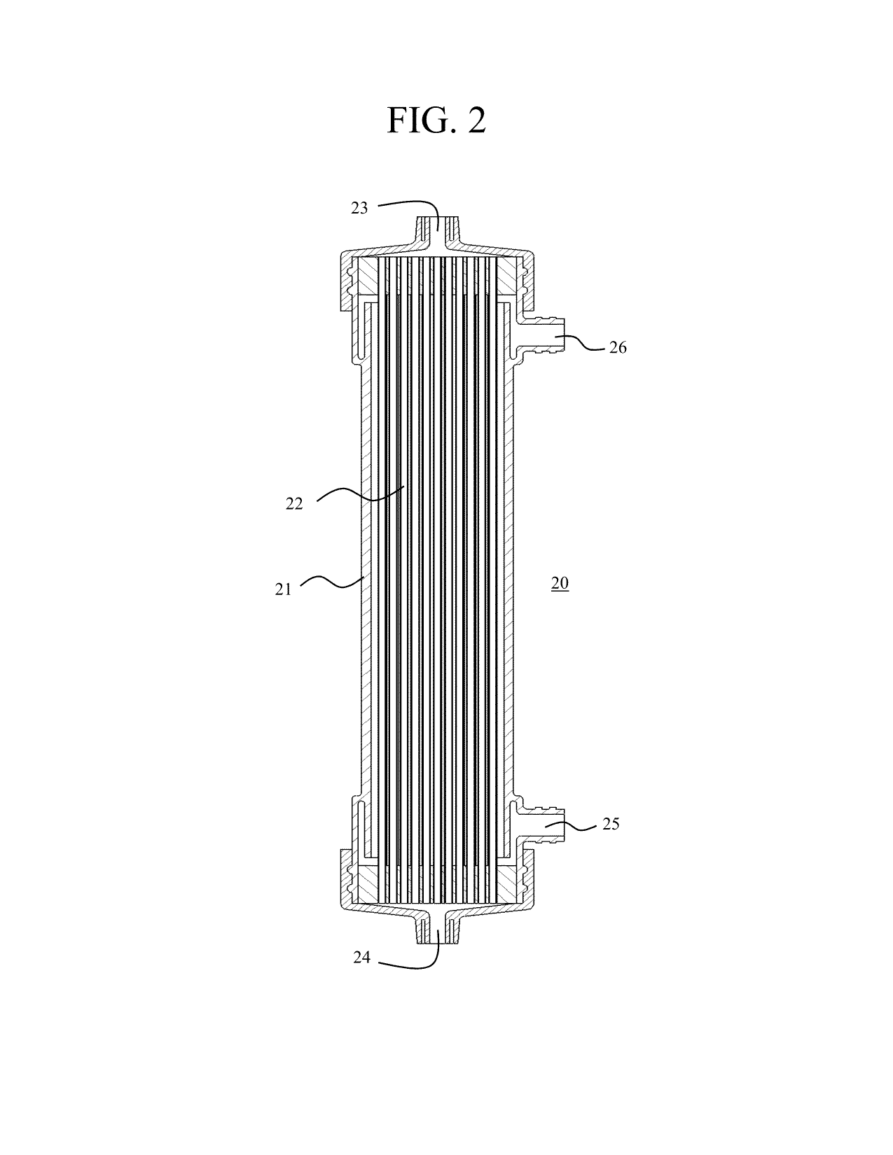

As shown in FIG. 2, the hemodialyzer 20 includes a hemodialyzer container 21 having an internal space and a hemodialysis membrane 22 accommodated in the internal space of the hemodialyzer container 21. The internal space of the hemodialyzer container 21 may be divided into a blood flow region and a dialysate flow region by the hemodialysis membrane 22. The hemodialyzer container 21 includes a blood inlet 23 disposed at one end thereof and a blood outlet 24 disposed at the other end thereof. Also, a dialysate inlet 26 and a dialysate outlet 25 may be provided on the outer surface of the hemodialyzer container 21. Blood passes through the blood flow region inside the hemodialyzer 20 and dialysate passes through outside the hemodialyzer. In this case, blood and dialysate may be desirably configured to flow in the opposite directions to each other.

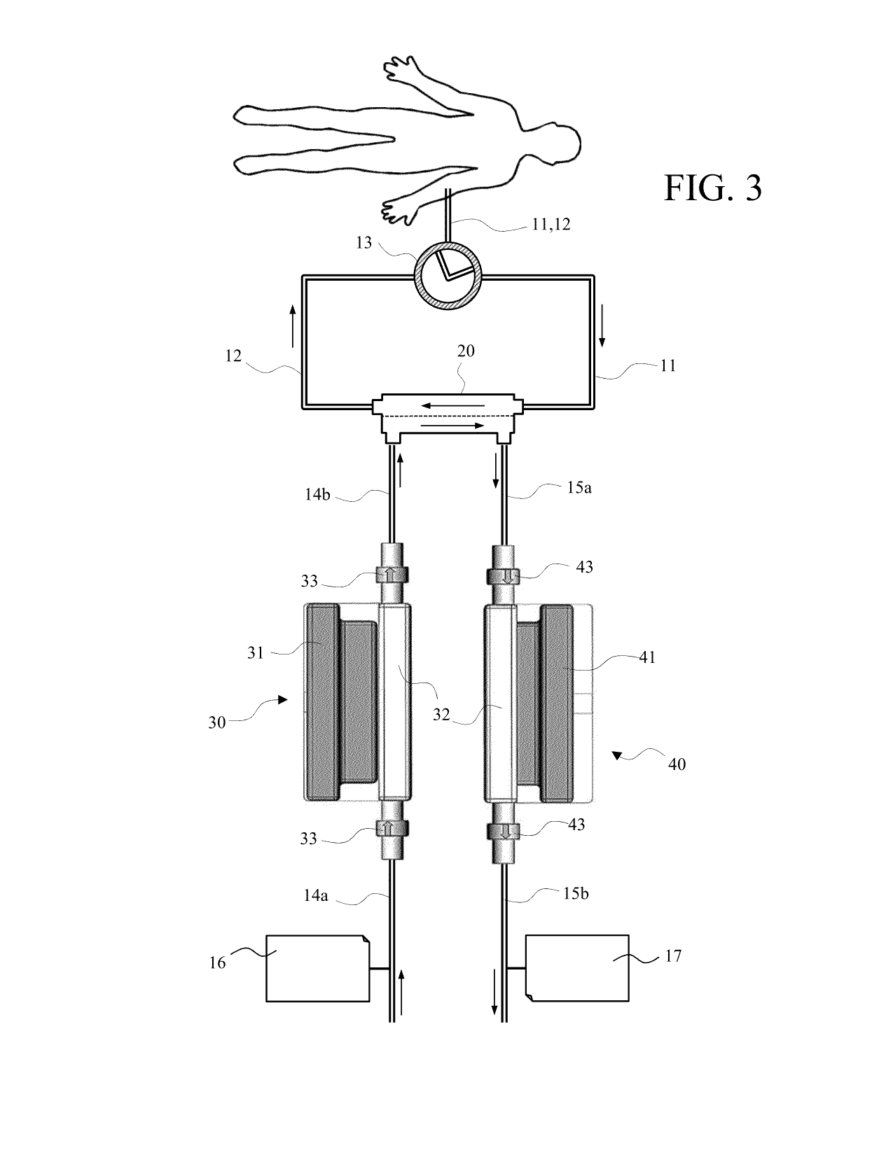

FIG. 3 illustrates the hemodialysis apparatus having the dialysate supply pump 30 and the dialysate discharge pump 40. The dialysate supply pump 30 may include a supply tube pressurizing member 31 which pressurizes a part of the dialysate supply tube 14 to transfer dialysate therein, a supporting wall 32 which supports the dialysate supply tube 14, and a supply tube one-way valve 33 provided at both sides of the dialysate supply tube pressurized by the supply tube pressurizing member 31 to prevent dialysate in the tube from flowing backward. In the same manner, the dialysate discharge pump 40 may include a discharge tube pressurizing member 41 which pressurizes a part of the dialysate discharge tube 15 to discharge dialysate therein, a supporting wall 32 which supports the dialysate discharge tube 15, and a discharge tube one-way valve 43 provided at both ends of the dialysate discharge tube 15 pressurized by the discharge tube pressurizing member 41 to prevent dialysate in the tube from flowing backward.

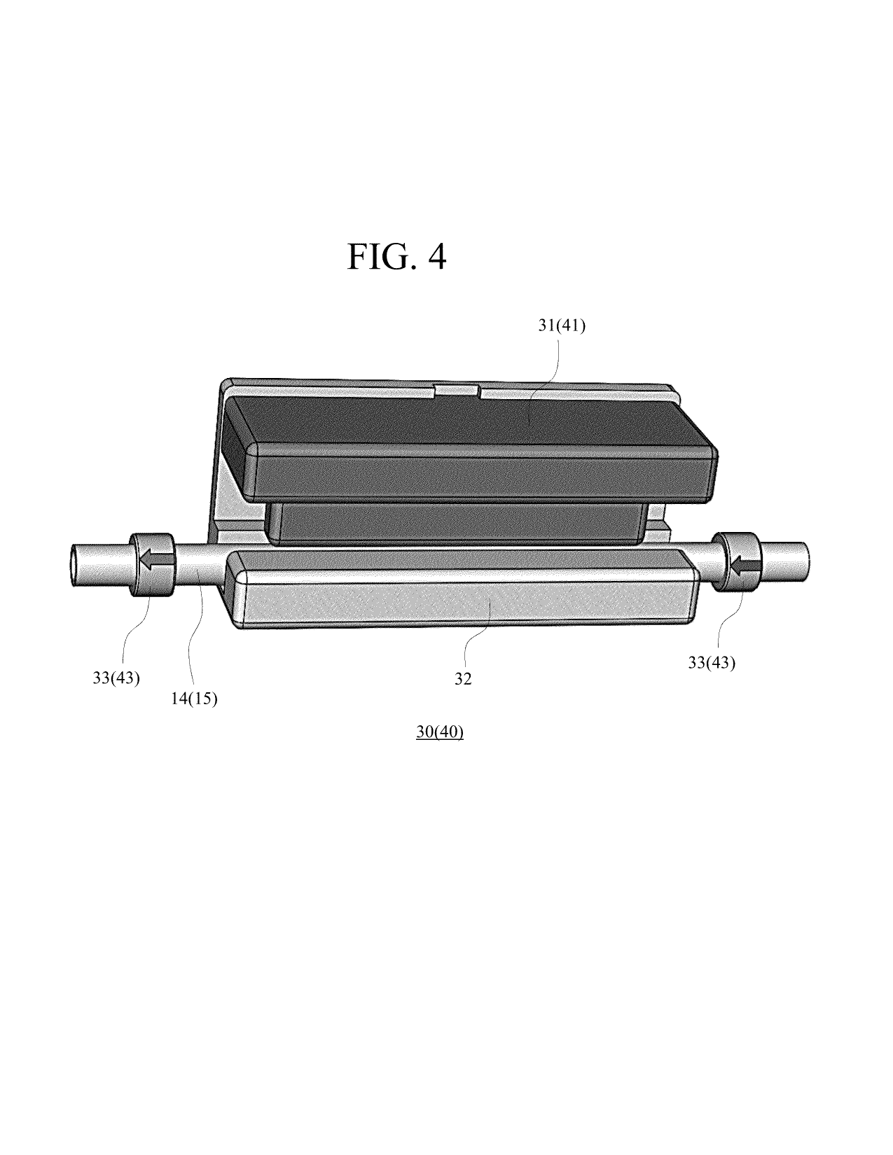

An enlarged view of the dialysate supply pump 30 and the dialysate discharge pump 40 having a tube pressurizing member 31 and 41, a supporting wall 32, and a tube one-way valve 33 and 43 is illustrated in FIG. 4. The dialysate supply tube 14 and the dialysate discharge tube 15 may be formed of a flexible material that can contract and expand.

The tube pressurizing member 31 or 41 pressurizes or expands the dialysate tube 14 or 15 while rectilinearly moving along a guide rail disposed on one side wall of the hemodialysis apparatus. The tube pressurizing member driver may include various structures that can apply a reciprocating movement force to the tube pressurizing member. An exemplary tube pressurizing member driver includes a cam for pressurizing the tube pressurizing member to the supporting wall 32 and a motor for rotating the cam. When the tube pressurizing member moves toward the tube supporting wall 32 due to the rotation of the cam, the dialysate tube is compressed. When an external force by the cam is removed, the tube pressurizing member 31 or 41 moves back to the original location, and the dialysate tube may be restored to the original state by its own elastic force, expanding the inner space thereof. The tube pressurizing member 31 and 41 are not limited to the structure shown in the drawing and may be modified into other structures that can compress and expand the dialysate tube 14 and 15. Also, the pressurizing member driver may be modified into another structure that can operate the tube pressurizing member.

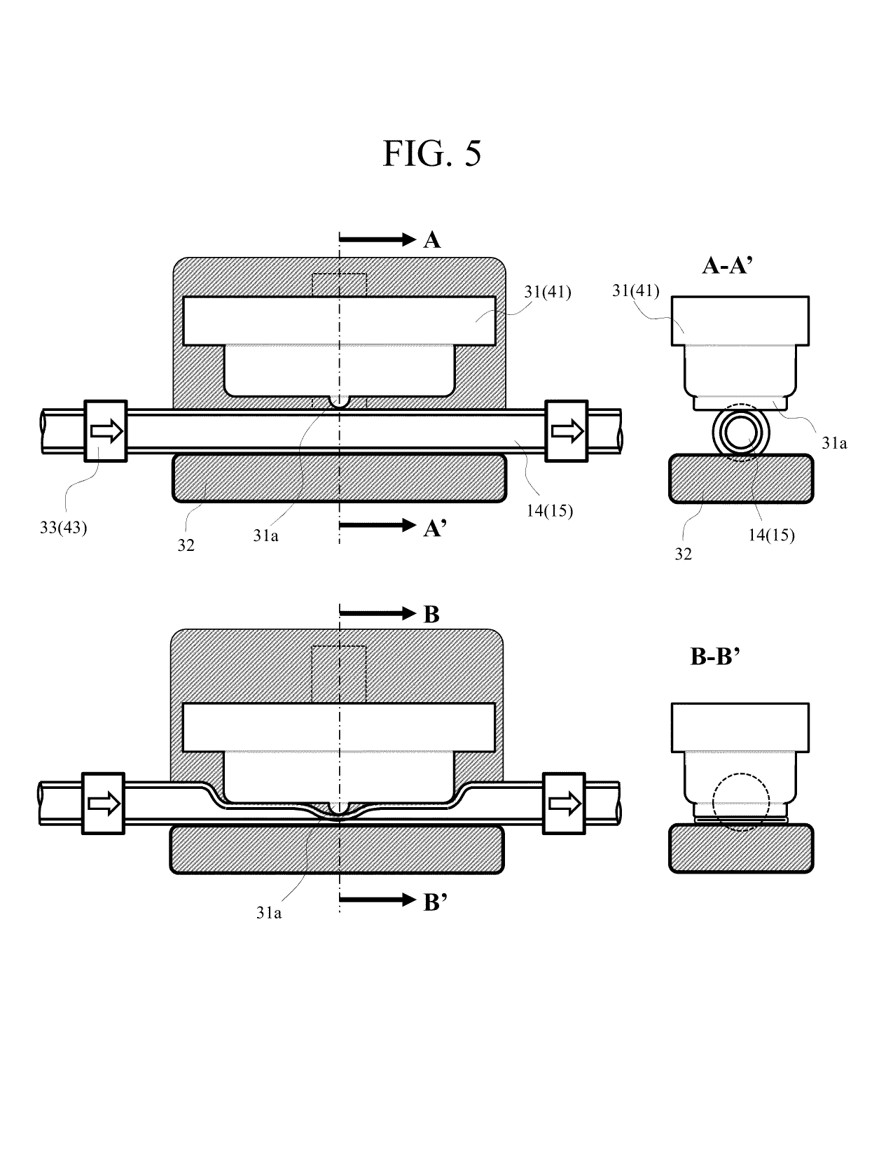

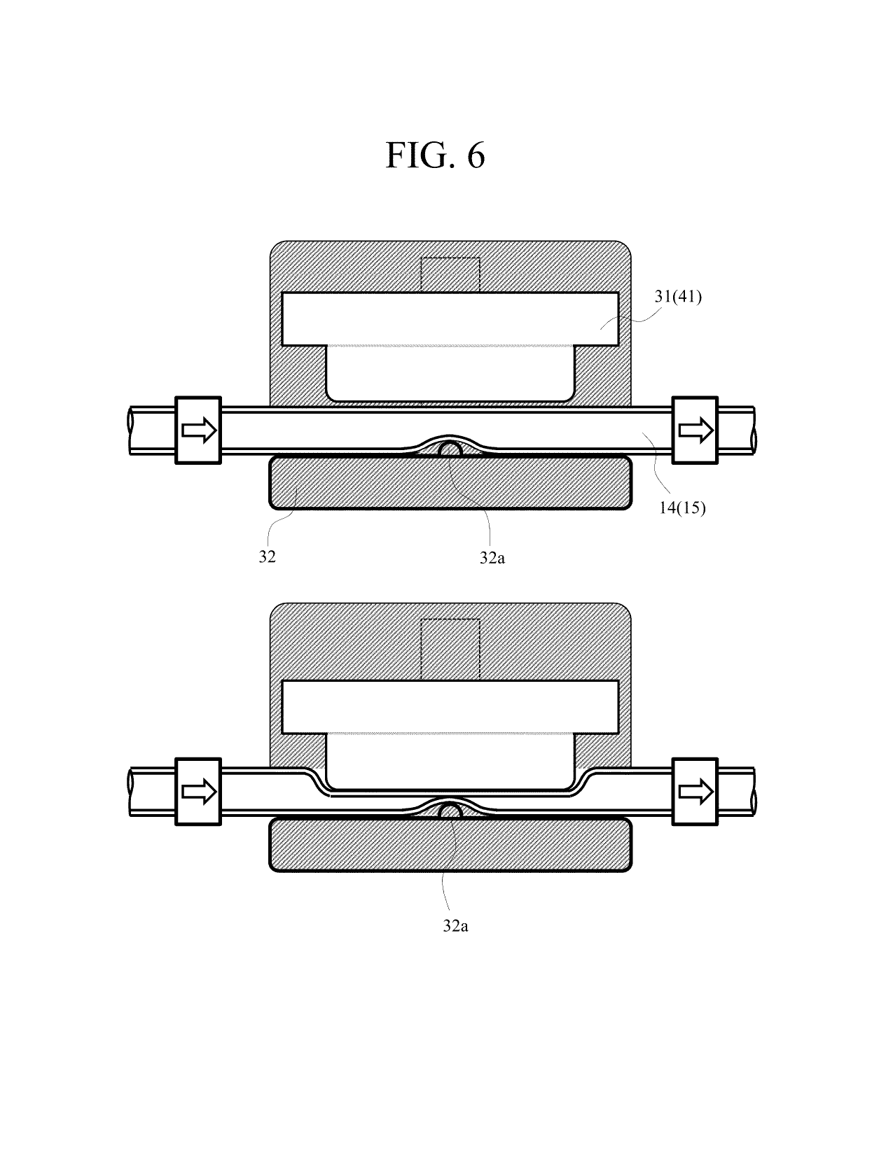

In this instance, dialysate flow passage through the dialysate tube 14 and 15 may be blocked when the dialysate tube is pressurized by the tube pressurizing member 31 and 41. For this, as shown in FIG. 5, the tube pressurizing member 31 and 41 may be provided with a tube pressurizing member protrusion 31a on the pressurizing surface of the tube pressurizing member. The tube pressurizing member pressurizes the dialysate tube, allowing the tube pressurizing member protrusion 31a to completely compress the dialysate tube, such that the flow passage through the dialysate tube can be blocked. Alternatively, as shown in FIG. 6, the supporting wall 32 may be provided with a supporting wall protrusion 32a on the surface of the supporting wall 32 which contacts with the dialysate tube 14 and 15 to enable the dialysate tube to be completed compressed and blocked.

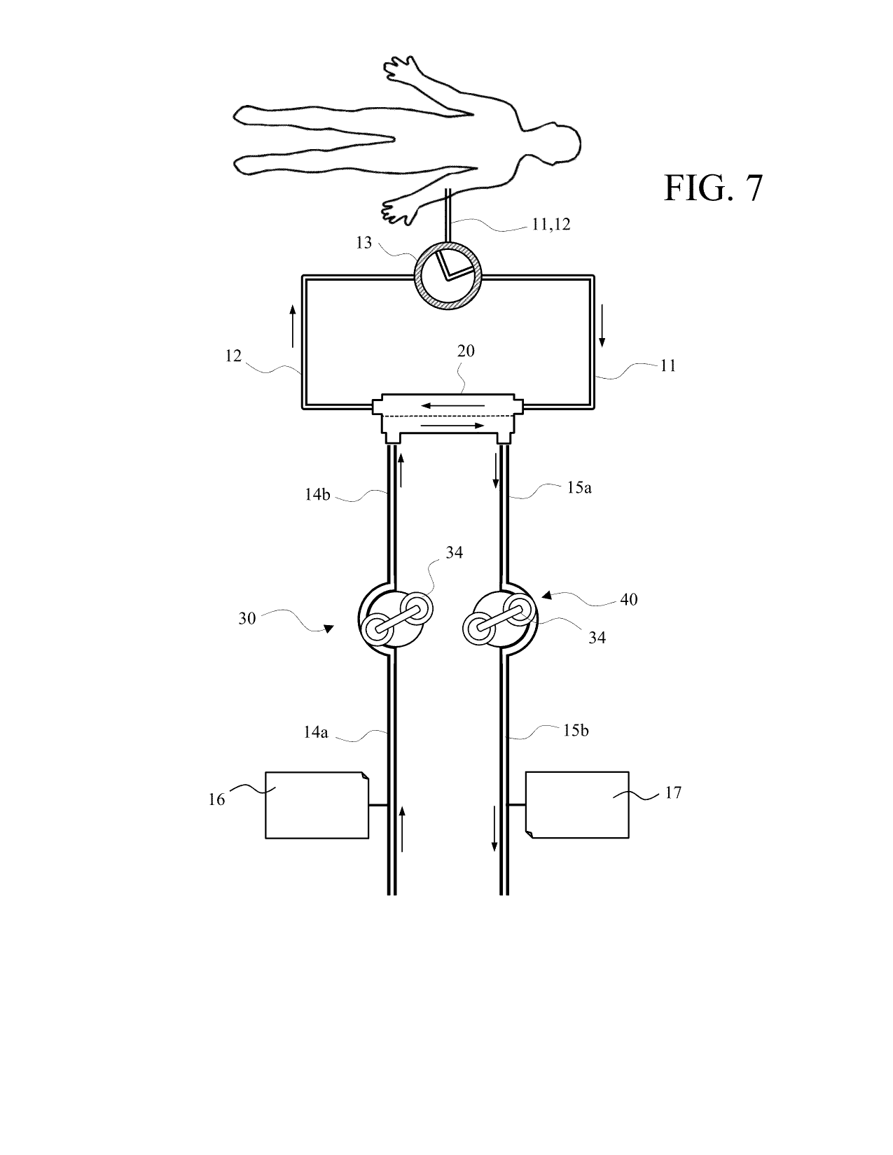

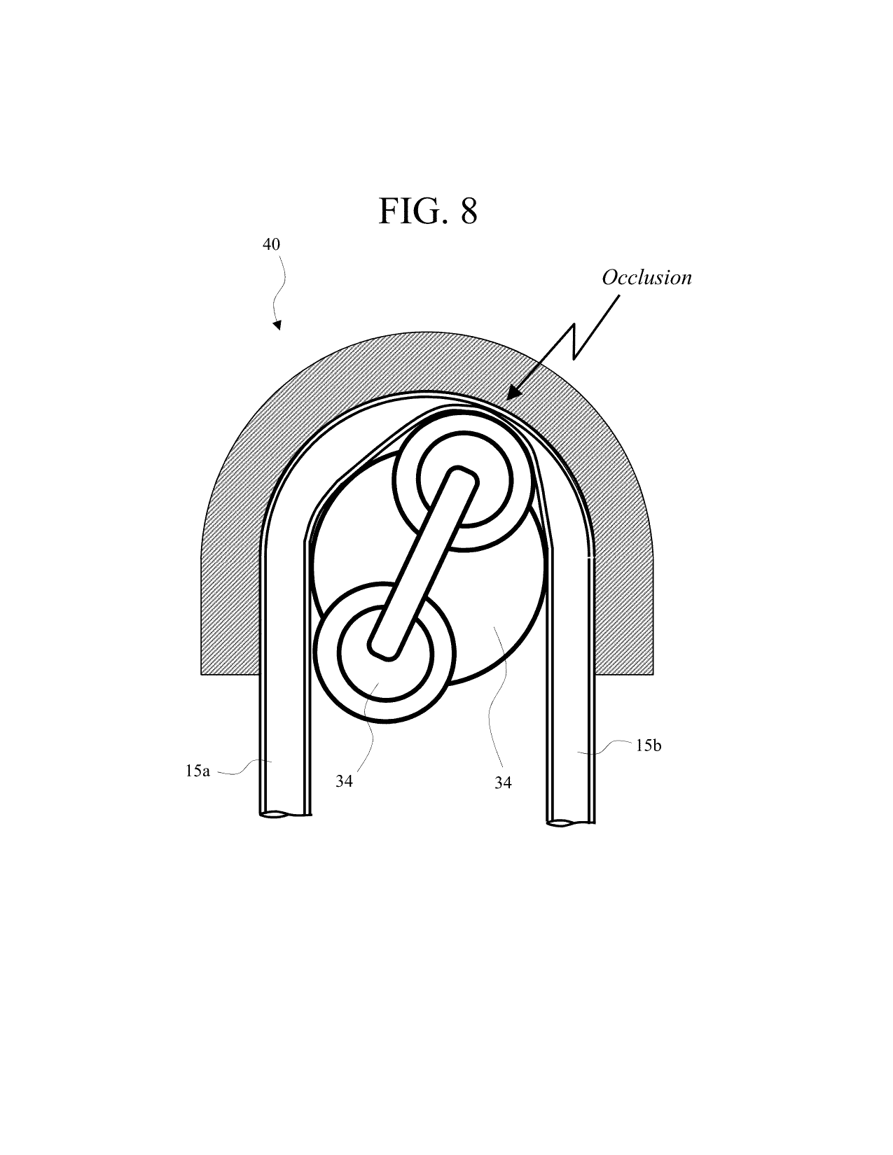

The dialysate supply pump 30 and the dialysate discharge pump 40 according to a first embodiment of the present invention is not limited to include a tube pressurizing member, a supporting wall, and a tube one-way valve. As shown in FIG. 7, the dialysate pumps 30 and 40 may be modified to have a structure including a roller 34 squeezing the dialysate tube to transfer dialysate therein and a roller driver to operate the roller 34. When dialysate is transferred by the rotational movement of the roller 34, the roller driver may use various structures to rotate the roller. Also, the roller may transfer dialysate by the linear movement thereof or by the sequential compression of the dialysate tube by a plurality of rollers. Dialysate flow passage through the dialysate tube 14 or 15 may be desirably blocked when the roller 34 compresses the dialysate tube, as illustrated in FIG. 8.

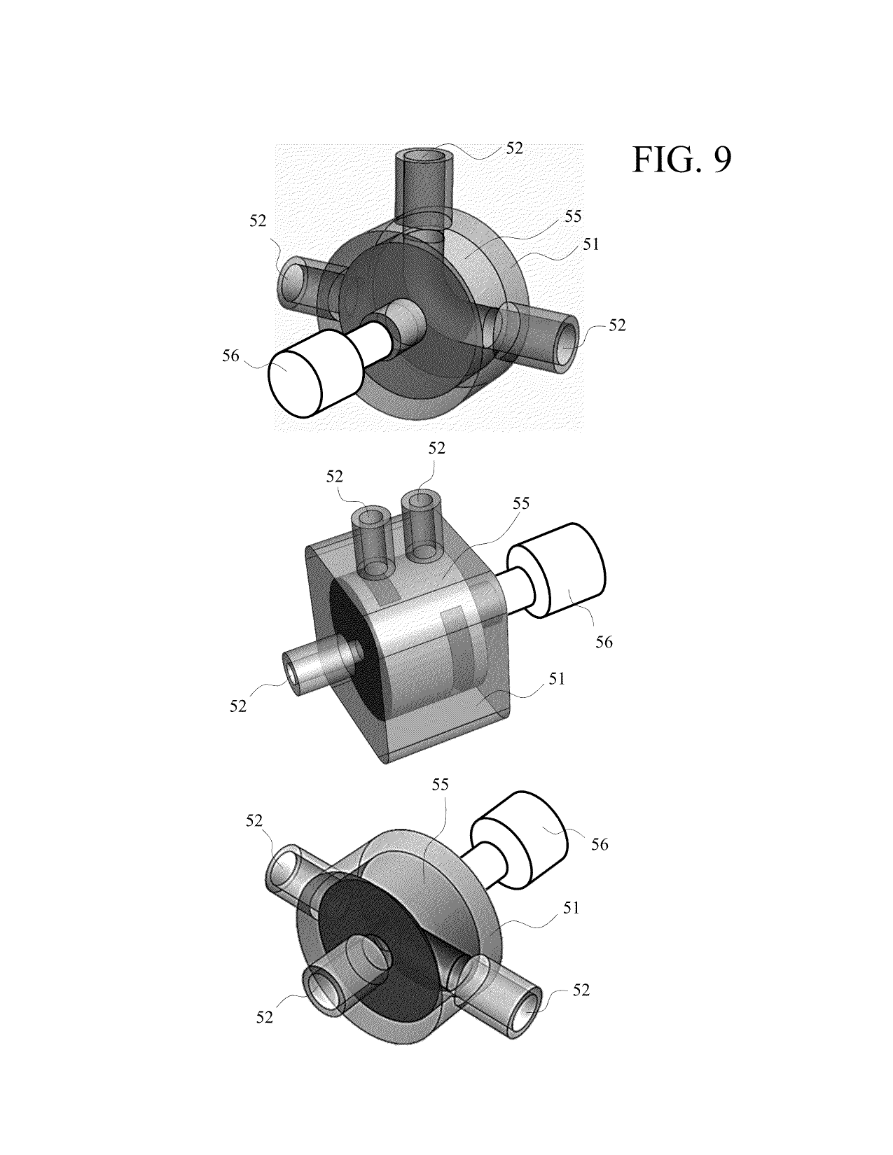

The blood flow controller 13 which controls blood flow passage through the blood tubes 11 and 12 may include a housing 51 having an internal space, a flow port 52 disposed on the housing, a rotor 55 disposed inside the housing and tightly attached into the inner surface of the housing 51 to connect flow passage between the flow ports 52, and a rotor driver for rotating the rotor 55, as illustrated in FIG. 9. Due to the rotation of the rotor 55, when a flow passage is connected between two flow ports 52, the flow passage through other flow ports that are not connected may be desirably blocked. The flow port 52 can be connected to a blood vessel of a patient, the first blood tube 11, and the second blood tube 12. The time for opening or blocking the flow passage is controlled by regulating the rotation speed of the rotor 55.

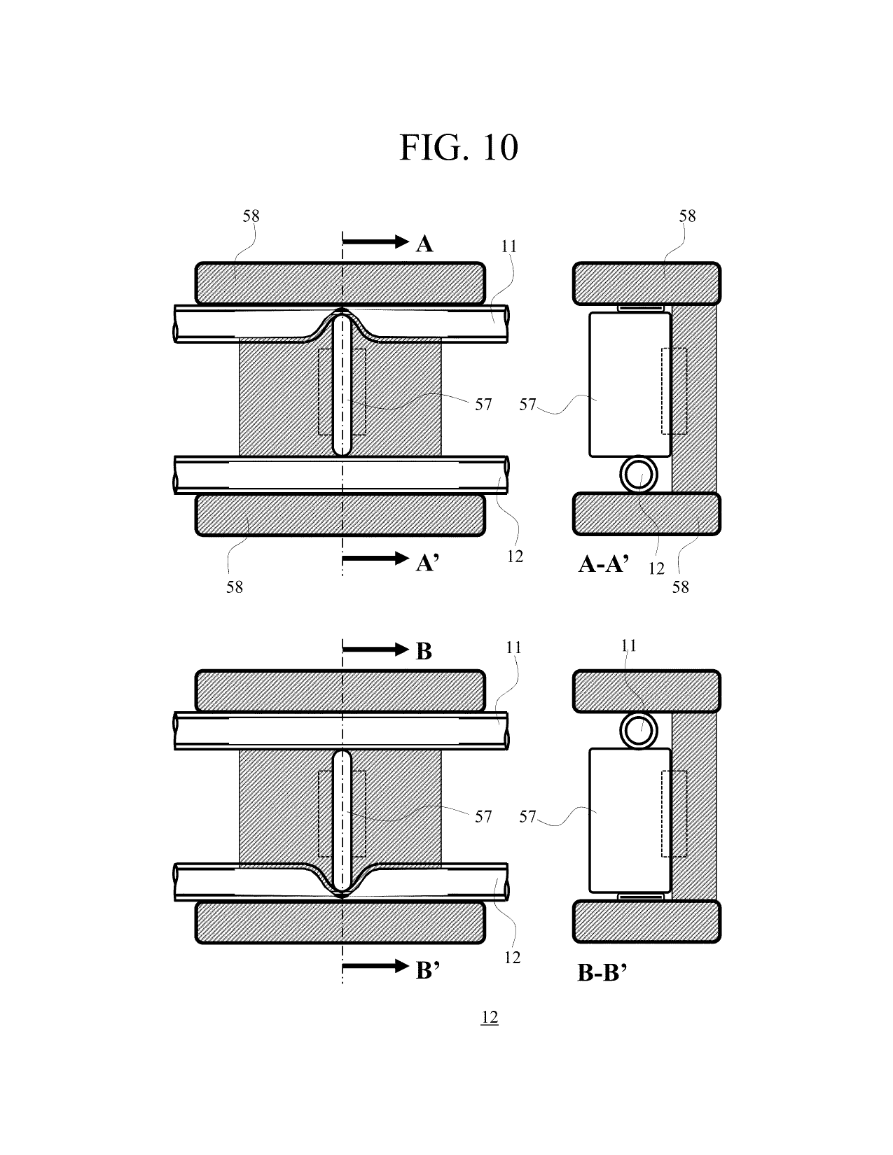

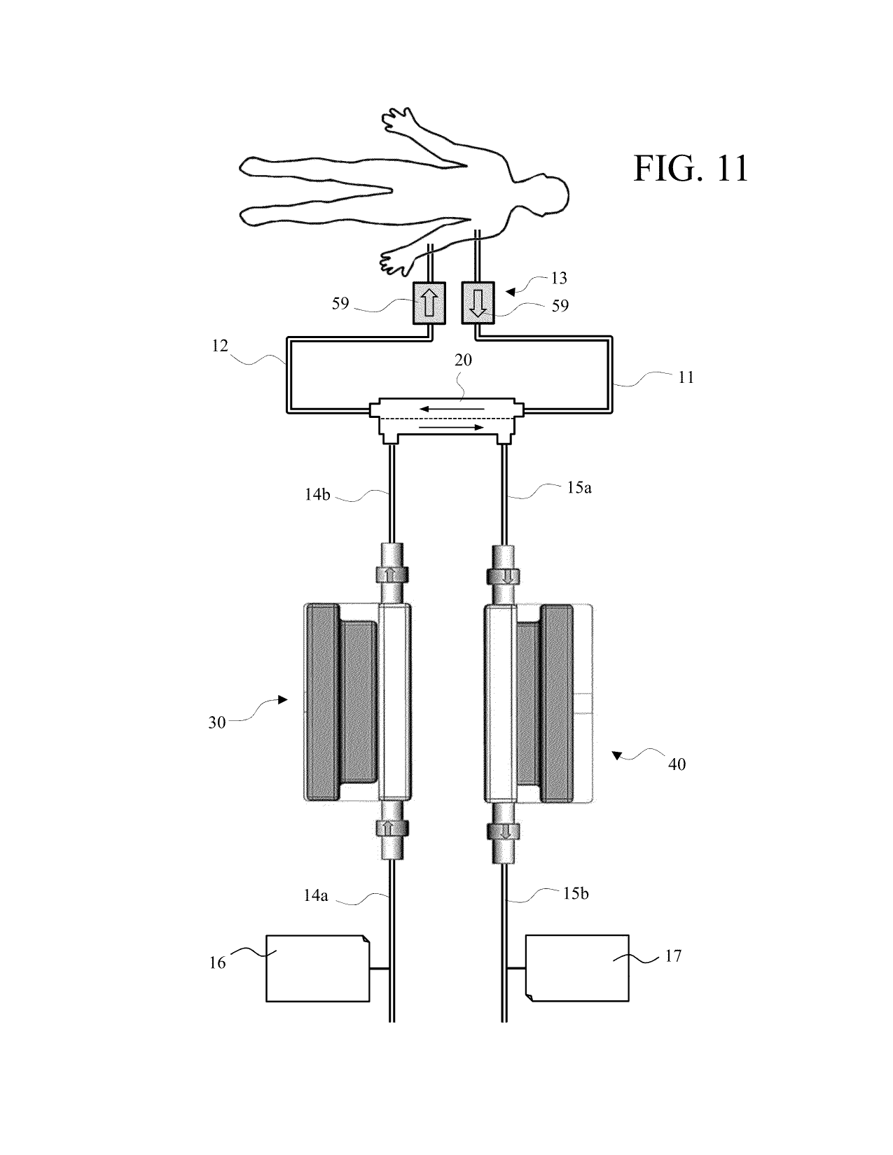

The blood flow controller 13 is not limited to the structures shown in FIG. 9 and may be modified into other structures that can open or block the blood flow passage through the blood tube 11 and 12. Another exemplary blood flow controller 13 is depicted in FIG. 10. The blood flow controller 13 may include a flow-blocking member 57 reciprocating in a straight line to compress the blood tube 11 or 12, a flow-blocking member driver providing a straight force to the flow-blocking member, and a flow-blocking wall 58 supporting the blood tube compressed by the flow-blocking member. When the flow-blocking member 57 moves to the first blood tube 11, one end of the flow-blocking member 57 compresses the tube supported by the flow-blocking wall 58 and blocks the blood flow passage therethrough. At this point, the blood flow passage through the second blood tube 12 is opened. Similarly, the flow-blocking member moves to the second blood tube 12 and the other end of the flow-blocking member 57 compresses the tube supported by the flow-blocking wall 58 and blocks blood flow therethrough.

In addition, the blood flow controller 13 may be modified into a structure in which a blood tube one-way valve 59 is disposed in the blood flow tube 11 and 12 to ensure blood to flow in a pre-determined direction. In other words, blood is supplied to the hemodialyzer 20 through the first blood tube 11 and then returned to a patient through the second blood tube 12 due to the blood tube one-way valves 59.

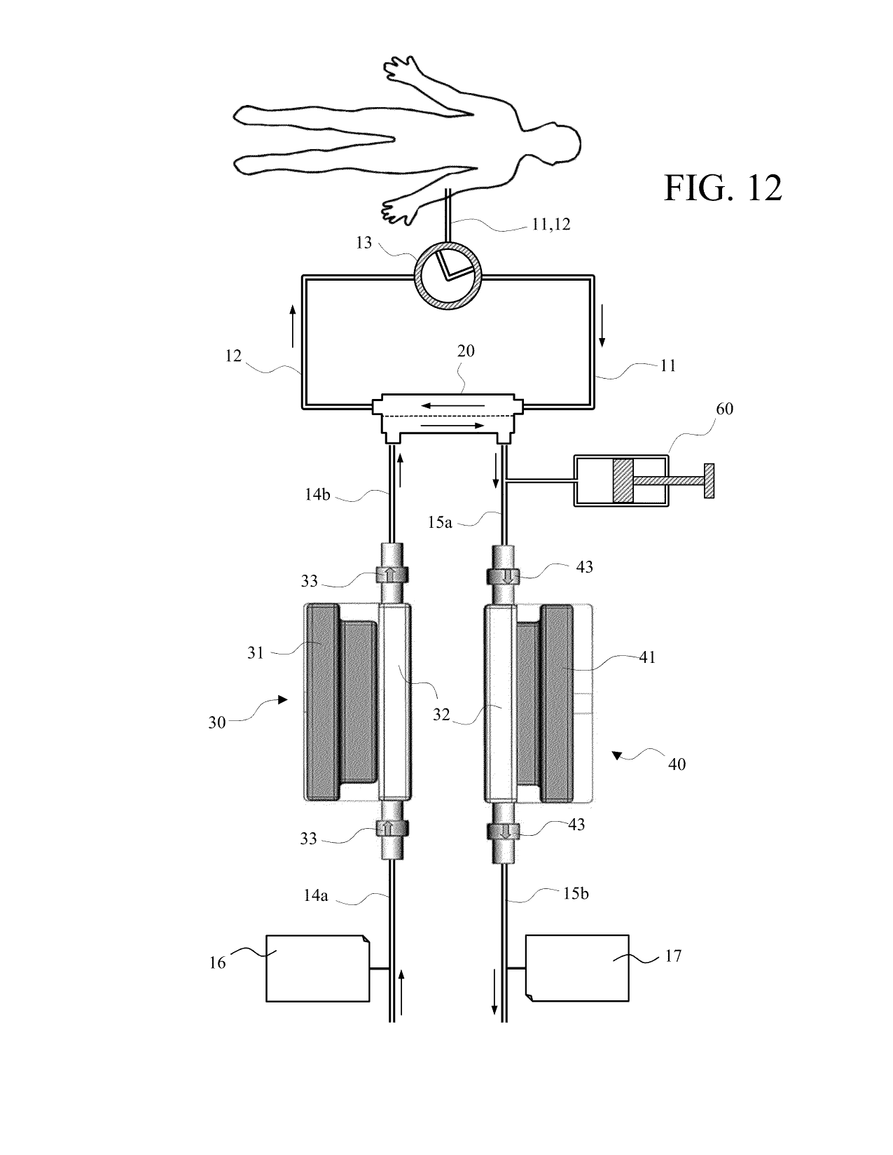

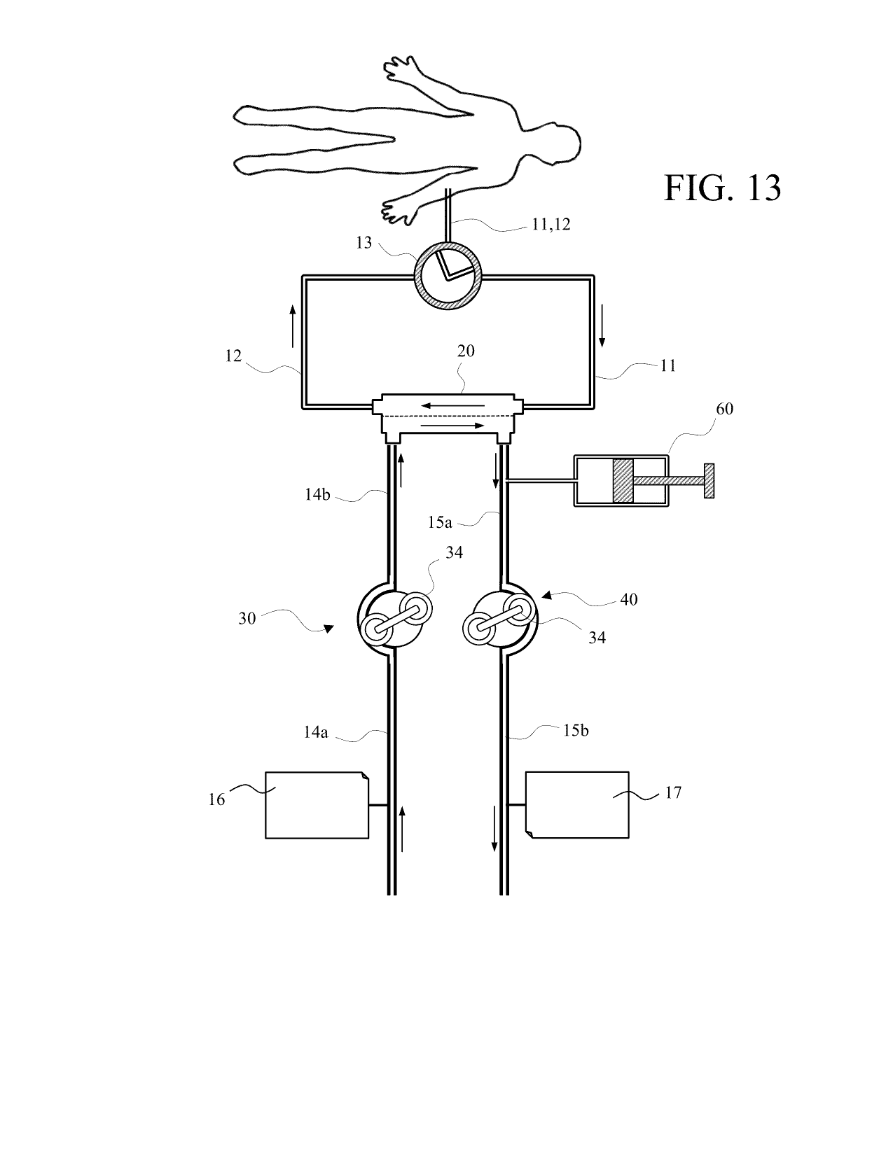

In addition, the hemodialysis apparatus 10 according to a first embodiment of the present invention may further include a volume chamber 60 to store dialysate, connected to the first dialysate discharge tube 15a. FIGS. 12 and 13 illustrate the hemodialysis apparatus 10 including the volume chamber 60. The internal space of the volume chamber 60 is expanded when dialysate flows in, while it is contracted when dialysate flows out. In FIGS. 12 and 13, the volume chamber has a structure having a container accommodating dialysate and a piston-shaped frame disposed inside the container, such that the piston-shape frame moves upward or downward when dialysate flows into or out of the container. The volume chamber is not limited in the structures shown in the drawings and may be modified into other structures. For example, the volume chamber may be changed to be connected to the second dialysate supply tube 14b.

Hereinafter, an operation of the hemodialysis apparatus 10 according to a first embodiment of the present invention will be described in detail with reference to the accompanying drawings.

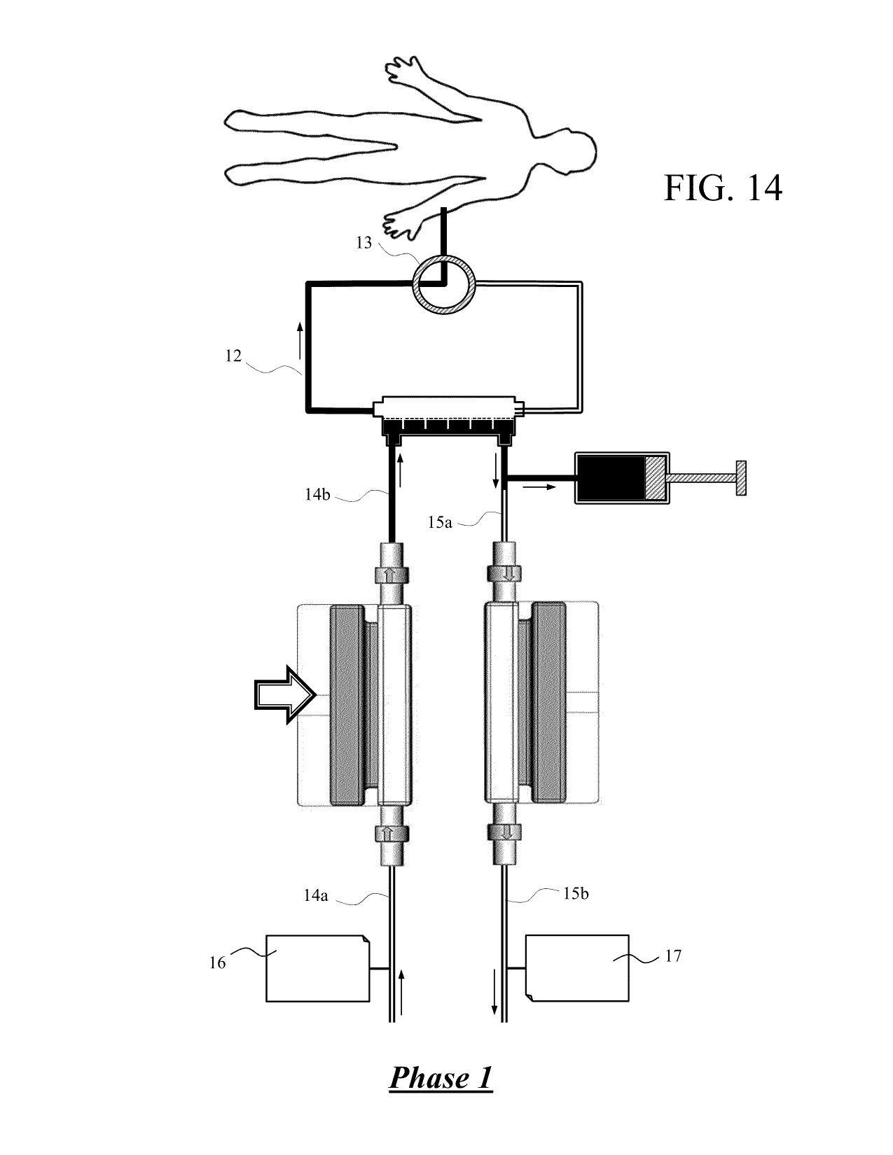

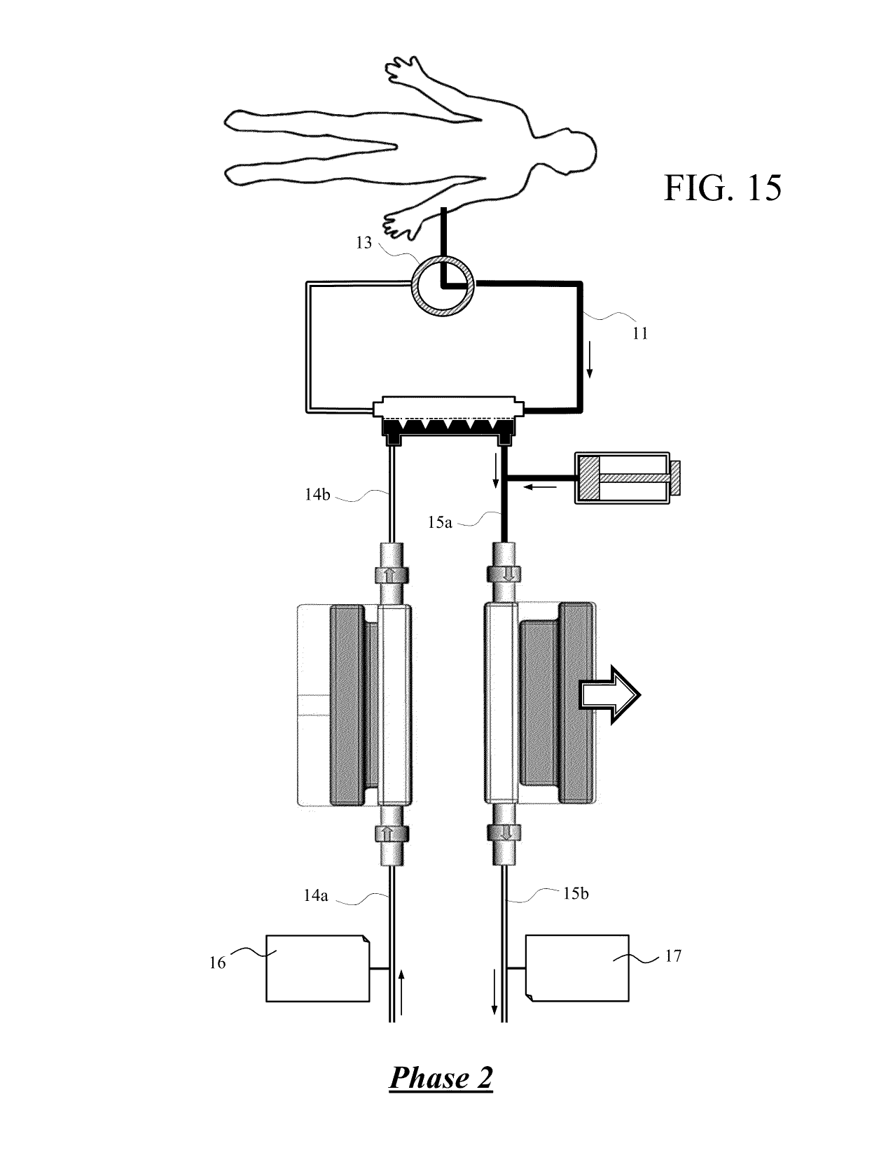

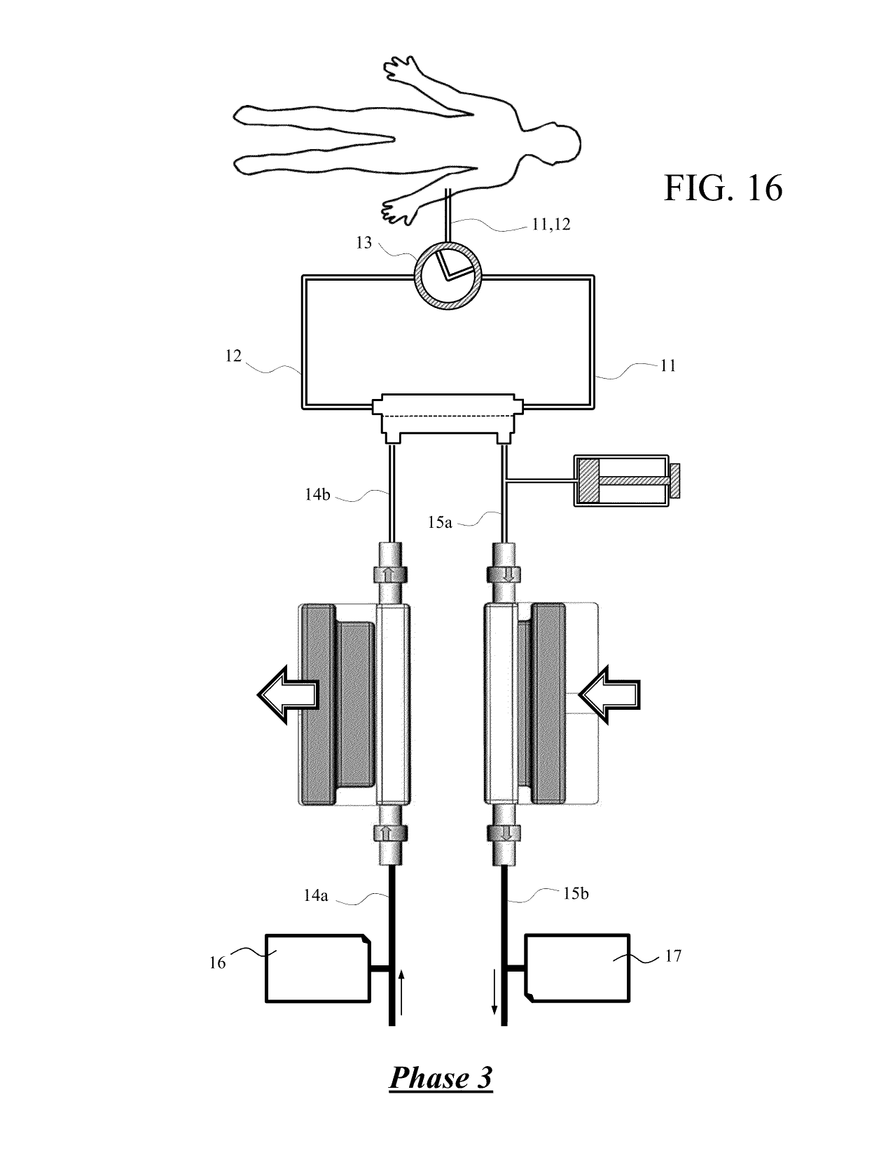

As shown in FIGS. 14 to 16, the flow of dialysate in the hemodialysis apparatus 10 according to a first embodiment of the present invention may include: a first phase where the dialysate supply tube 14 is pressurized by the supply tube pressurizing member 31 and the dialysate therein is supplied to the hemodialyzer 20, while the dialysate discharge tube 15 is pressurized and blocked by the discharge tube pressurizing member 41; a second phase where the dialysate discharge tube 15 is expanded by the discharge tube pressurizing member 41 and the dialysate of the hemodialyzer is transferred to the dialysate discharge tube 15 expanded, while the dialysate supply tube 14 is pressurized and blocked by the supply tube pressurizing member 31; and a third phase where the dialysate supply tube 14 is filled with fresh dialysate due to the expansion of the dialysate supply tube 14 by the supply tube pressurizing member 31 and the dialysate of the dialysate discharge tube 15 is discarded therefrom due to the compression of the dialysate discharge tube 15 by the discharge tube pressurizing member 41. The dialysate flow in the hemodialysis apparatus 10 according to a first embodiment of the present invention repeats the first, second and third phases.

During the first phase, dialysate inside the dialysate supply tube 14 is transferred to the hemodialyzer 20 and at this time, the discharge tube 15 is blocked by the discharge tube pressurizing member 41. Thus, the hydraulic pressure of the dialysate flow region inside the hemodialyzer 20 increases compared to the pressure of the blood flow region, and backfiltration in which water in dialysate moves toward the blood flow region occurs. At this time, since the blood controller 13 opens a blood flow passage through the second blood tube 12 and blocks a blood flow passage through the first blood tube 11, blood of the hemodialyzer is returned to a patient due to the backfiltration.

In contrast, during the second phase, dialysate of the hemodialyzer 20 is transferred to the dialysate discharge tube 15 that is expanded by the discharge tube pressurizing member 41 and at this time, the dialysate supply tube 14 is blocked by the supply tube pressurizing member 31. Thus, the pressure of the dialysate flow region inside the hemodialyzer 20 decreases compared to the pressure of the blood flow region, and filtration in which water in blood moves toward the dialysate flow region occurs. Furthermore, at this moment the blood controller 13 opens a blood flow passage through the first blood tube 11 and blocks a blood flow passage through the second blood tube 12, and thus blood of a patient is supplied to the hemodialyzer through the first blood tube 11 due to the filtration. That is, a cycle of the expansion and compression of the dialysate supply pump 30 and the dialysate discharge pump 40 configures a cycle of filtration and backfiltration inside the hemodialyzer 20 and simultaneously enables blood of a patient to be supplied to the hemodialyzer 20 and then returned. Water and waste products are removed during the filtration and lost water is supplemented during the backfiltration.

Here, a flow rate of blood supplied to the hemodialyzer 20 (QB1) and returned to a patient (QB2) can be calculated. QB1 and QB2 may be regarded to be equal to the amount of filtration and backfiltration inside the hemodialyzer. Blood tubes 11 and 12 may have a fixed volume despite the change in the pressure therein. QB1 and QB2 may be expressed by Equation (1) using a compression-expansion volume of the dialysate supply tube 14 (Vd), a compression-expansion volume of the dialysate discharge tube 15 (Ve), and a volume of the volume chamber 60 (Vc).

QB1=Ve−Vc,QB2=Vd−Vc(ml/stroke) (1)

Accordingly, the blood flow rates QB1 and QB2 can be maintained lower than the dialysate flow rates due to the use of the volume chamber 60 and thus diffusive hemodialysis efficiency can be enhanced.

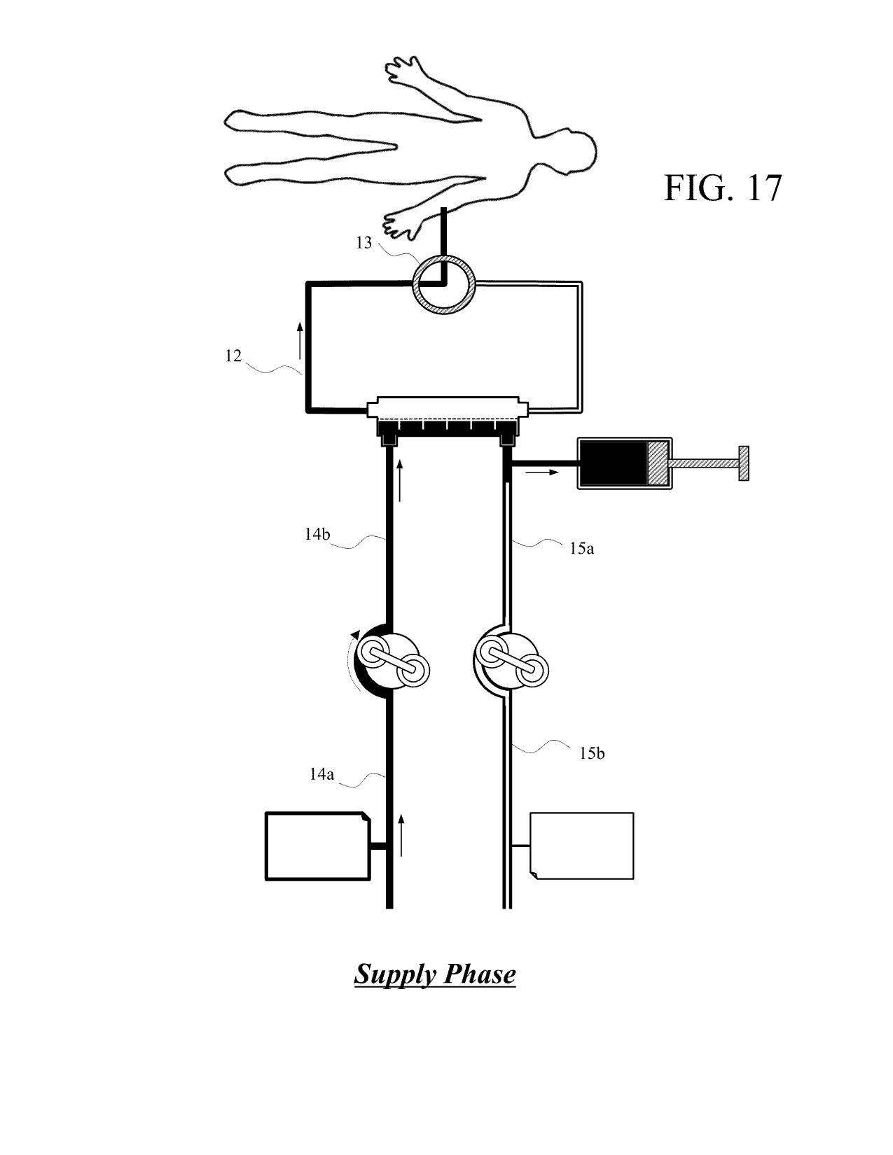

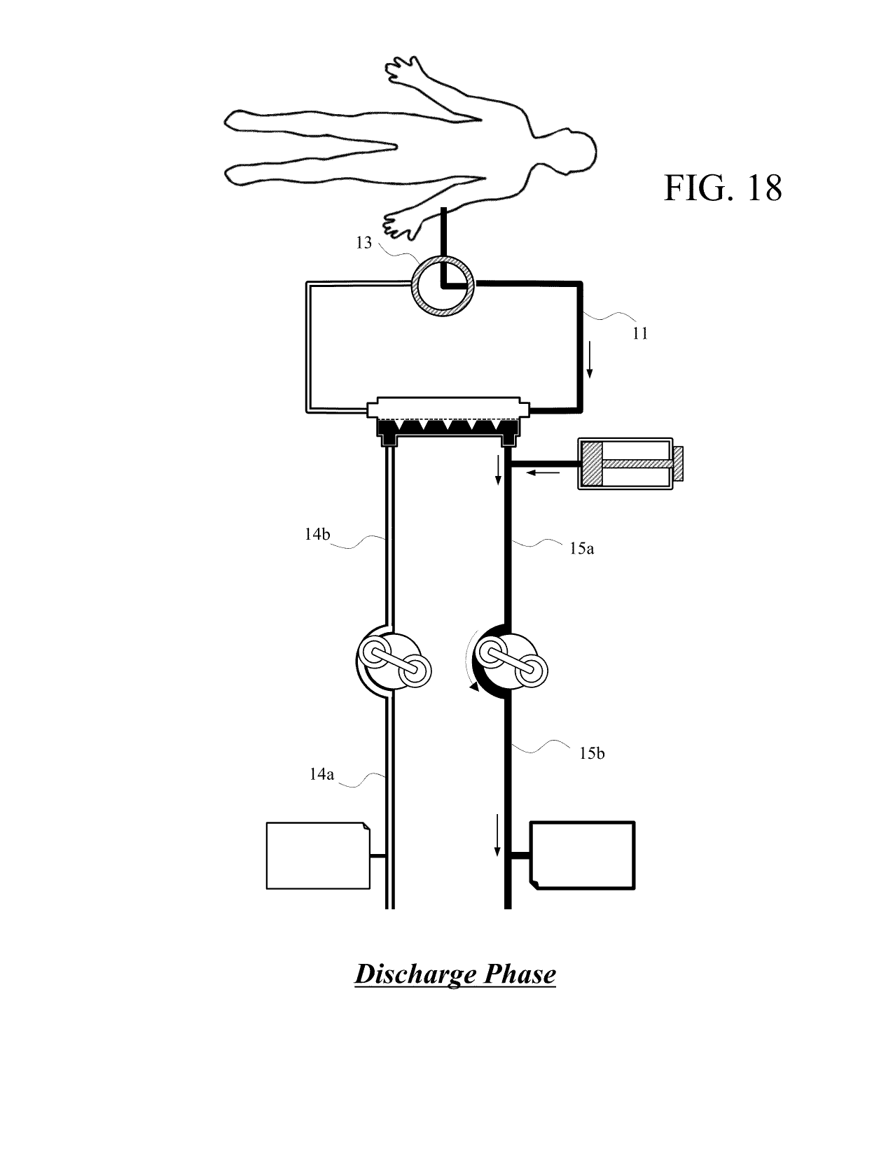

FIGS. 17 and 18 are views showing the operation of the hemodialysis apparatus 10 in which the dialysate supply pump 30 and the dialysate discharge pump 40 include a roller 34 and a roller driver. In this instance, the dialysate flow includes: a supply phase in which a flow rate of dialysate that is supplied to the hemodialyzer 20 by the dialysate supply pump 30 is larger than the flow rate of dialysate discharged from the hemodialyzer by the dialysate discharge pump 40; and a discharge phase in which the flow rate of dialysate that is supplied to the hemodialyzer by the dialysate supply pump 30 is smaller than the flow rate of dialysate that is discharged from the hemodialyzer by the dialysate discharge pump 40.

The blood flow controller 13 opens a blood flow passage through the second blood tube 12 and blocks a blood flow passage through the first blood tube 11 during the supply phase, whereas the blood flow controller opens the first blood tube and blocks the second blood tube during the discharge phase.

During the supply phase, since the amount of dialysate supplied to the hemodialyzer 20 is larger than that discharged from the hemodialyzer, the hydraulic pressure of the dialysate flow region inside the hemodialyzer 20 increases compared to the pressure of the blood flow region, leading to the backfiltration inside the hemodialyzer. Dialysate may be stored in the volume chamber 60 and blood is returned to a patient through the second blood tube 12 during the supply phase. On the other hand, the pressure conditions inside hemodialyzer are reversed during the discharge phase, such that a filtration occurs inside the hemodialyzer and blood of a patient is supplied to a hemodialyzer through the first blood tube 11. As described above, the flow rates of blood supplied to or returned from the hemodialyzer 20 can be calculated using the stroke volume of the dialysate supply pump 30 and the dialysate discharge pump 40 and the volume of the volume chamber 60 in a similar way.

Hereinafter, a hemodialysis apparatus 10 according to a second embodiment of the present invention and an operation thereof will be described in detail with reference to the accompanying drawings.

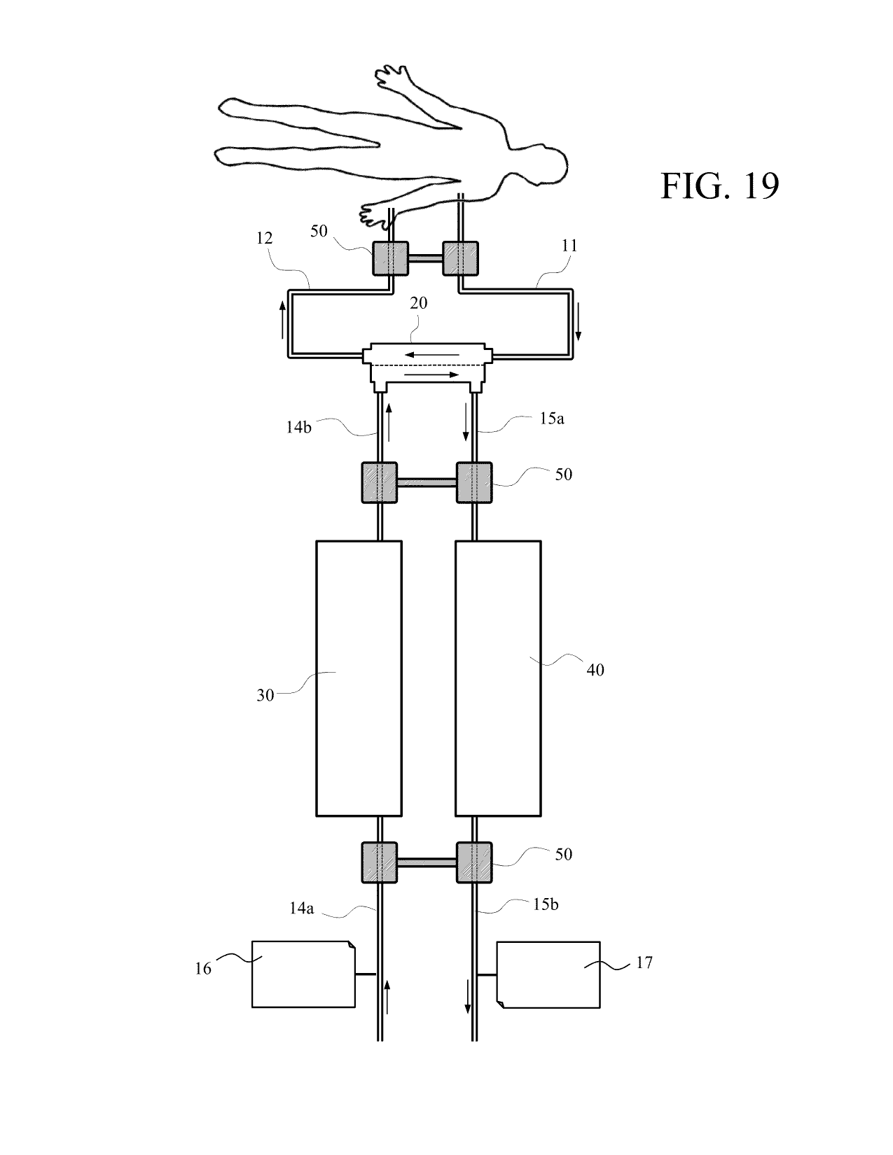

As shown in FIG. 19, a hemodialysis apparatus 10 according to a second embodiment of the present invention includes the hemodialyzer 20 in which a mass transfer occurs between blood and dialysate, the first blood tube 11 supplying blood of a patient to the hemodialyzer 20, the second blood tube 12 returning blood of the hemodialyzer to a patient, a flow control device 50 controlling blood and dialysate flow passage through the blood tube 11 or 12 and the dialysate tube 14a, 14b, 15a or 15b, a dialysate supply pump 30 supplying dialysate to the hemodialyzer 20, and a dialysate discharge pump 40 discharging dialysate having passed the hemodialyzer.

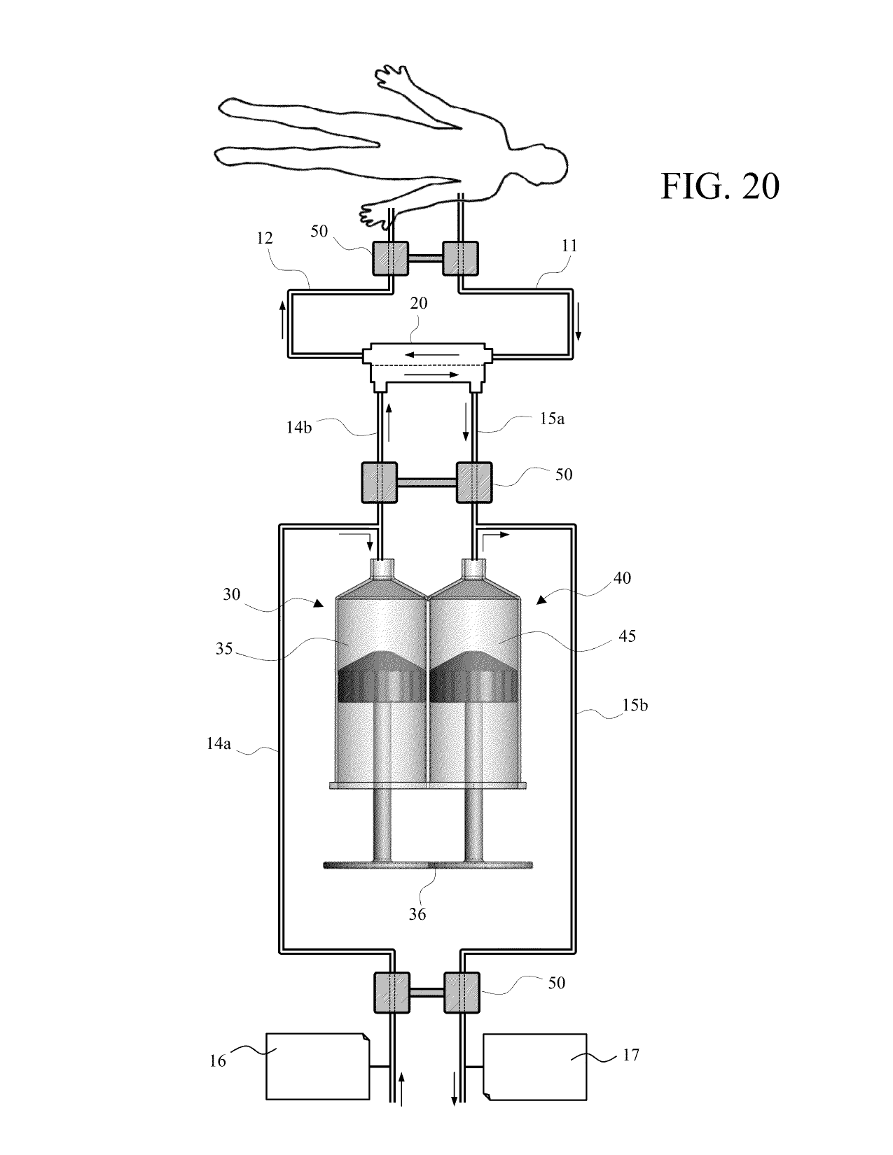

FIG. 20 illustrates the dialysate supply pump 30 and the dialysate discharge pump 40 according to a second embodiment of the present invention. The dialysate supply pump 30 and the dialysate discharge pump 40 include a cylinder 35 and 45 having an internal space, a piston 36 disposed inside the cylinder, and a piston driver allowing the piston to reciprocate. The piston driver includes various structures that can compress or expand the supply pump cylinder 35 and the discharge pump cylinder 45 by pushing or pulling the piston 36. The supply pump cylinder 35 and the discharge pump cylinder 45 are simultaneously compressed or expanded and thus a single piston and a piston driver may be used to compress or expand the cylinders 35 and 45.

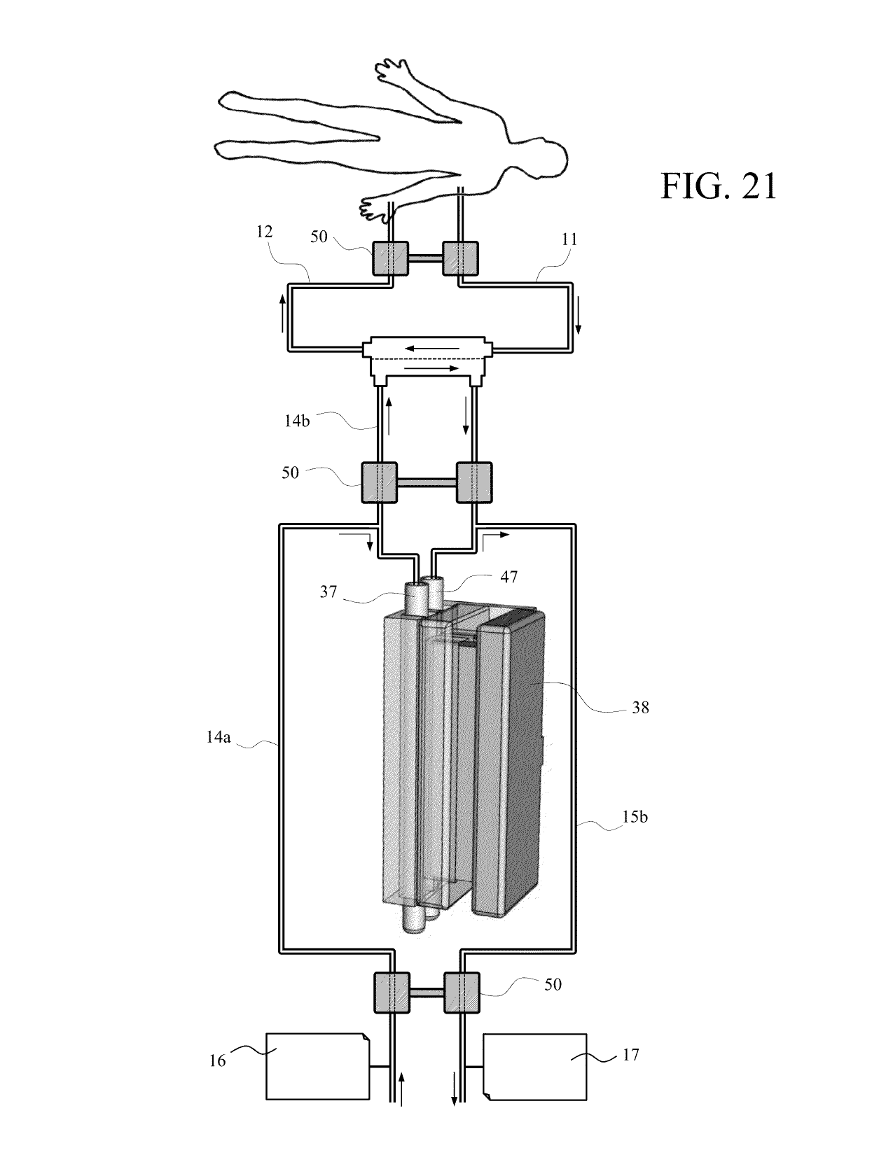

The dialysate supply pump 30 and the dialysate discharge pump 40 according to a second embodiment of the present invention are not limited to the structure described in FIG. 20. Another example of the dialysate supply pump 30 and the dialysate discharge pump 40 is illustrated in FIG. 21. The dialysate supply and discharge pumps 30 and 40 may have a sac 37 and 47 having an internal space, formed of a flexible material than can contract and expand, a sac pressuring member 38 that pressurizes the sac 37 and 47, and a sac pressurizing member driver allowing the sac pressurizing member to reciprocate. Likewise, the supply pump sac 37 and the discharge pump sac 47 are simultaneously compressed or expanded, and thus a single sac pressurizing member 38 and a sac pressurizing member driver may be used to compress or expand the sacs 37 and 47.

The sac pressurizing member 38 compresses or expands the supply pump sac 37 and the discharge pump sac 47 while rectilinearly moving along a guide rail disposed on one side wall. The sac pressurizing member driver may include various structures that can apply a reciprocating movement force to the sac pressurizing member 38. An exemplary sac pressurizing member driver includes a cam for pressurizing the sac pressurizing member 38 and a motor for rotating the cam. The sac pressurizing member 38 may be modified into other structures that can compress and expand the supply pump sac 37 and the discharge pump sac 47.

The flow control device 50 controls the flow passage through the blood tube 11 and 12, the dialysate supply tube 14a and 14b, and the dialysate discharge tube 15a and 15b. Blood is supplied to the hemodialyzer 20 through the first blood tube 11, while dialysate is supplied to the dialysate pumps 30 and 40 through the first dialysate supply tube 14a and the first dialysate discharge tube 15a. On the contrary, blood is returned to a patient through the second blood tube 12 and dialysate is discharged out of the dialysate pumps 30 and 40 through the second dialysate supply tube 14b and the second dialysate discharge tube 15b. In this instance, the flow control device 50 according to a second embodiment of the present invention may alternately block the first blood tube 11, the first dialysate supply tube 14a, and the first dialysate discharge tube 15a, or the second blood tube 12, the second dialysate supply tube 14b, and the second dialysate discharge tube 15b.

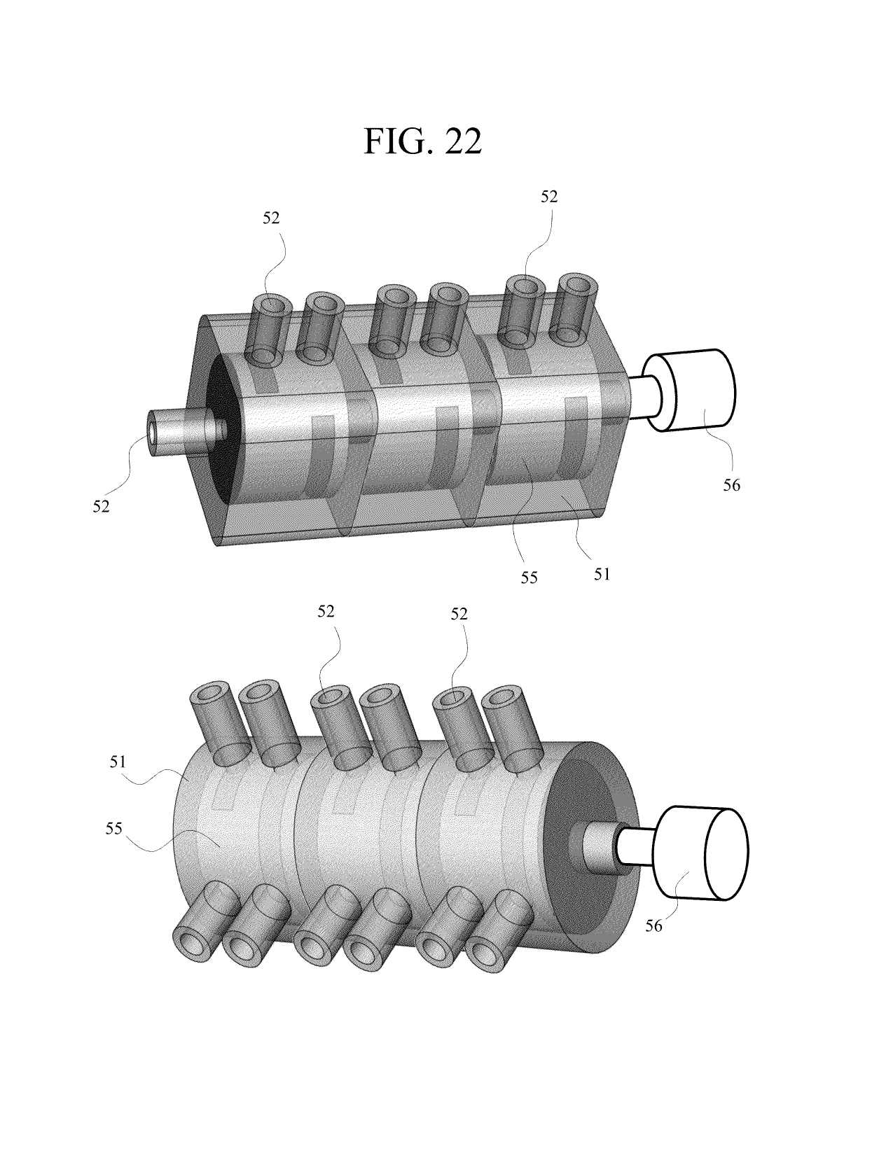

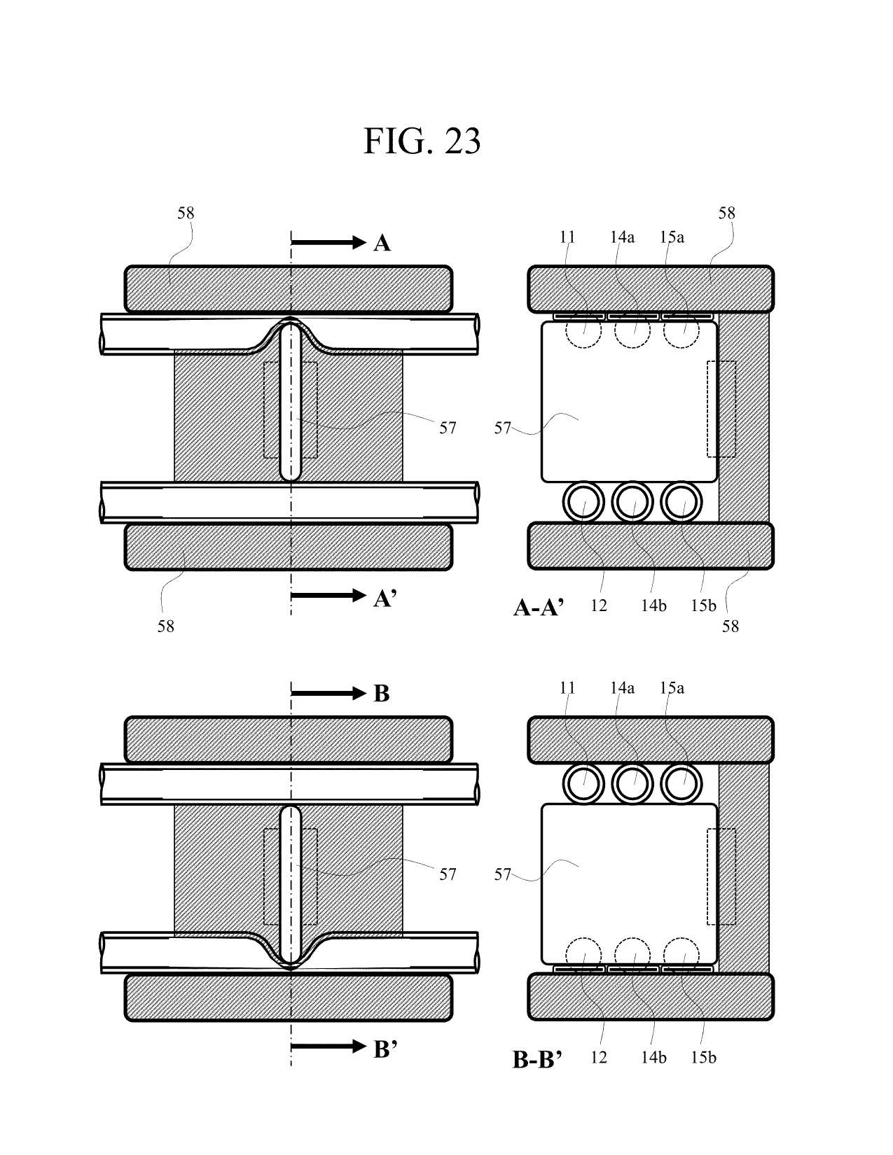

FIGS. 22 and 23 illustrate exemplary views of the flow control device 50. As shown in FIG. 22, the flow control device 50 may include a housing 51 having an internal space, a flow port 52 disposed on the housing, a rotor 55 disposed inside the housing to connect flow passage between the flow ports 52, and a rotor driver rotating the rotor 55. Due to the rotation of the rotor 55, when flow passages are connected between some of the flow ports 52, flow passages through other flow ports that are not connected are desirably blocked. The flow port 52 can be connected to a blood vessel of a patient, the blood tube 11 and 12 and the dialysate tube 14a, 14b, 15a and 15d. The time for opening or blocking the flow passage can be controlled by regulating the rotation speed of the rotor 55.

Alternatively, as shown in FIG. 23, the flow control device 50 may have a structure that includes a flow-blocking member 57 reciprocating in a straight line to compress the blood and dialysate tubes, a flow-blocking member driver providing a straight force to the flow-blocking member 57, and a flow-blocking wall 58 supporting the blood and dialysate tubes compressed by the flow-blocking member. When the flow-blocking member 57 moves to a side of the first blood tube 11, the first dialysate supply tube 14a, and the first dialysate discharge tube 15a, one end of the flow-blocking member 57 compresses the tubes 11, 14a and 15a supported by the flow-blocking wall 58 and blocks the flow passage through the tubes. At this moment, the flow passage through the second blood tube 12, the second dialysate supply tube 14b and the second dialysate discharge tube 15b remains open. Similarly, the flow-blocking member moves to the opposite side and the other end of the flow-blocking member 57 compresses the second blood tube 12, the second dialysate supply tube 14b and the second dialysate discharge tube 15b, blocking flow therethrough.

The structure of the flow control device is not limited to that shown in the drawings, and may be modified into other structures that can alternately block the first blood tube 11, the first dialysate supply tube 14a, and the first dialysate discharge tube 15a, or the second blood tube 12, the second dialysate supply tube 14b, and the second dialysate discharge tube 15b.

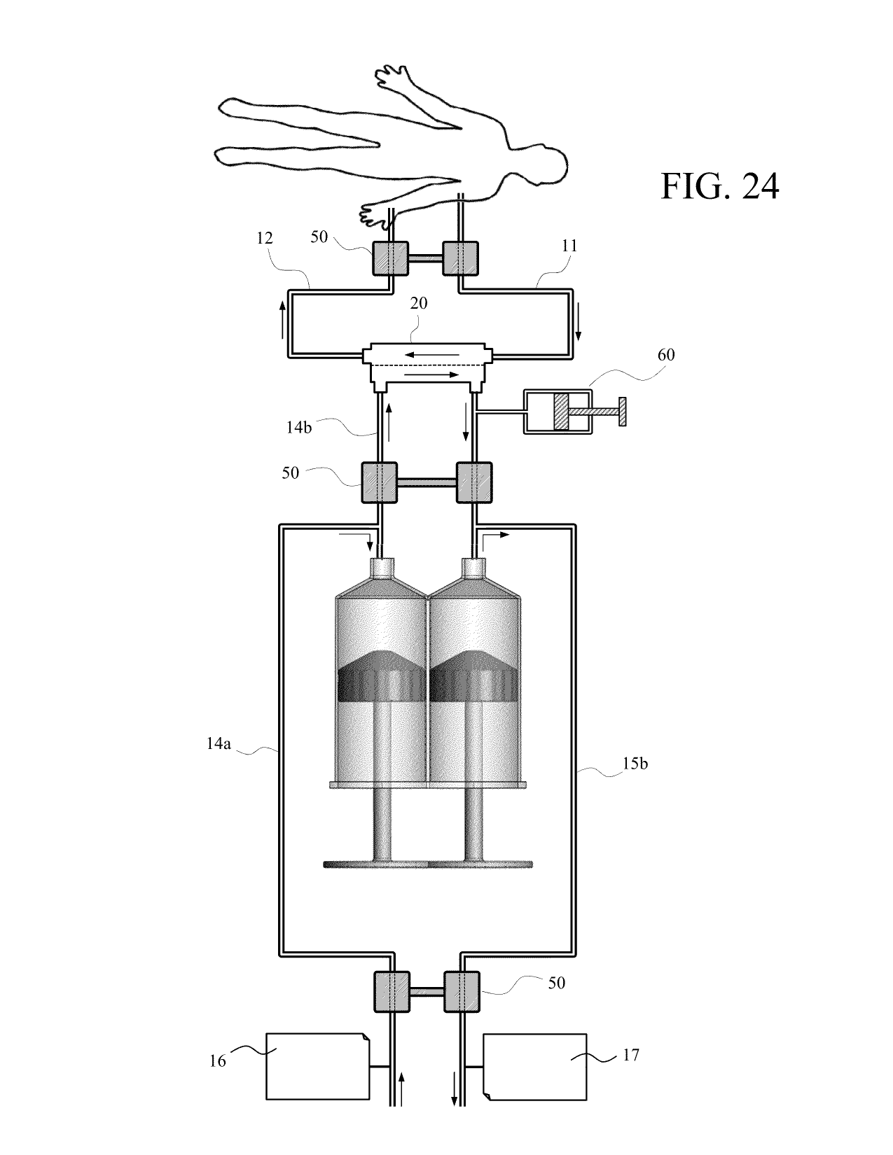

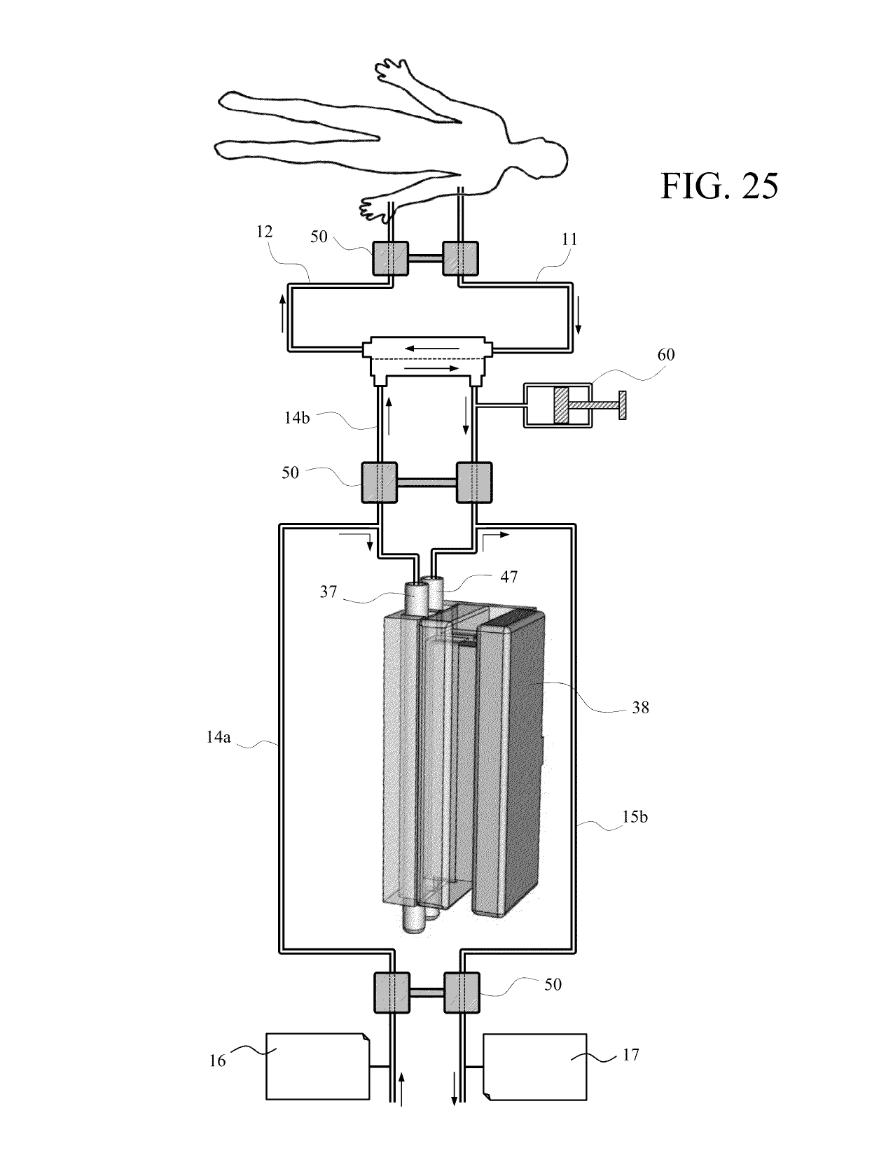

In the same manner, the hemodialysis apparatus 10 according to a second embodiment of the present invention may also have the volume chamber 60 to store dialysate. FIGS. 24 and 25 illustrates the hemodialysis apparatus 10 including the volume chamber 60 according to a second embodiment of the present invention. The structure and operation of the volume chamber 60 are same as that described in the hemodialysis apparatus 10 according to the first embodiment of the present invention.

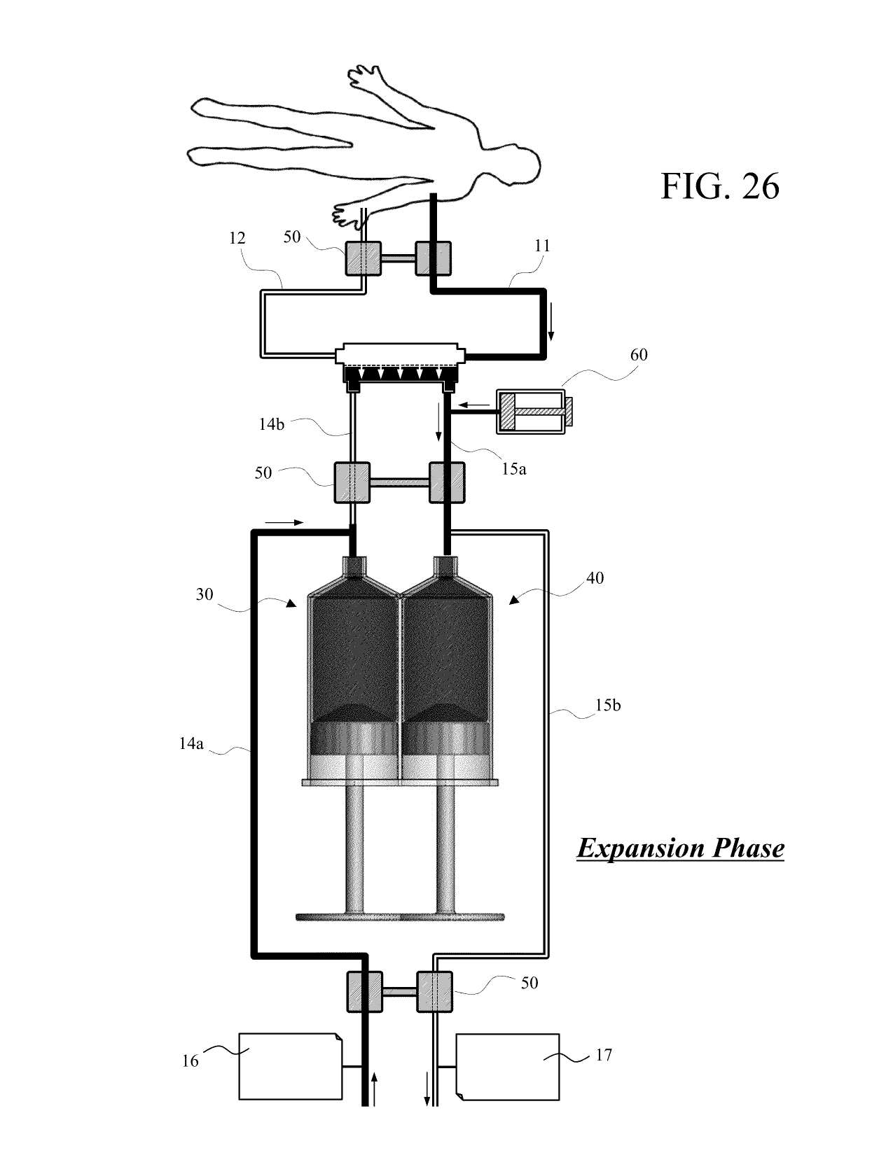

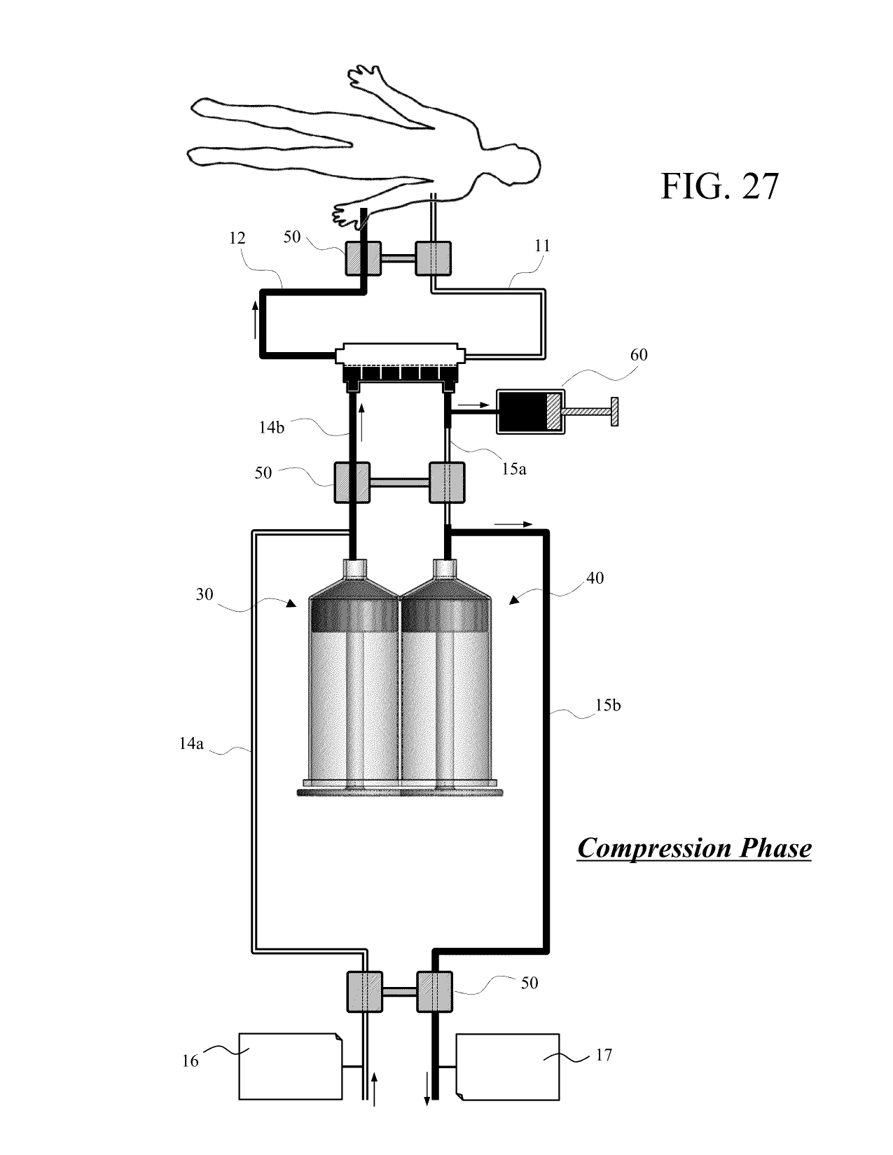

FIGS. 26 and 27 illustrate the operation of the hemodialysis apparatus 10 according to a second embodiment of the present invention. The hemodialysis apparatus 10 repeats an expansion phase in which the cylinders 35 and 45 or the sacs 37 and 47 are expanded by the pulling of the piston 36 and a compression phase in which the cylinders or sacs are compressed.

As shown in FIG. 26, when the supply pump cylinder 35 and the discharge pump cylinder 45 are expanded, the flow control device 50 opens the first blood tube 11, the first dialysate supply tube 14a and the first dialysate discharge tube 15a, and blocks the second blood tube 12, the second dialysate supply tube 14b and the second dialysate discharge tube 15b. Due to the expansion of the supply pump cylinder 35, the internal pressure of the cylinder is reduced and dialysate flows into the cylinder 35. In this case, since the second dialysate supply tube 14b is blocked by the flow control device 50, dialysate does not counter flow from the hemodialyzer 20 to the supply pump cylinder. Similarly, due to the expansion of the discharge pump cylinder 45, dialysate of the hemodialyzer flows into the discharge pump cylinder. In this case, since the second dialysate discharge tube 15b is blocked, dialysate of the dialysate recovery tank 17 does not counter flow to the discharge pump cylinder 45. Simultaneously, since the second dialysate supply tube 14b is blocked, the hydrostatic pressure of the dialysate flow region inside the hemodialyzer is lowered and a filtration in which water and uremic toxin in blood move to the dialysate flow region occurs. The filtration enables blood of a patient to be supplied to the hemodialyzer 20 through the first blood tube 11 that is opened. In addition, dialysate may be stored in the volume chamber 60 during this phase.

On the other hand, as shown in FIG. 27, when the supply pump cylinder 35 and the discharge pump cylinder 45 are compressed, the flow control device 50 blocks the first blood tube 11, the first dialysate supply tube 14a and the first dialysate discharge tube 15a, and opens the second blood tube 12, the second dialysate supply tube 14b and the second dialysate discharge tube 15b. Due to the compression of the discharge pump cylinder 45, dialysate of the discharge pump cylinder 45 is discharged out of the discharge pump cylinder 45. In this case, since the first dialysate discharge tube 15a is blocked, dialysate does not counter flow to the hemodialyzer 20. Similarly, when the supply pump cylinder 35 is compressed, dialysate inside the cylinder is supplied to the hemodialyzer 20. Since the first dialysate supply tube 14a is blocked, dialysate in the cylinder 35 does not counter flow to the direction of the dialysate supply tank 16. Simultaneously, since the first dialysate discharge tube 15a is blocked, the hydrostatic pressure of the dialysate flow region inside the hemodialyzer 20 increases and a backfiltration occurs. Backfiltration enables blood of the hemodialyzer to be returned to a patient through the second blood tube 12 that is opened. Dialysate may be discharged out of the volume chamber 60 during this compression phase.

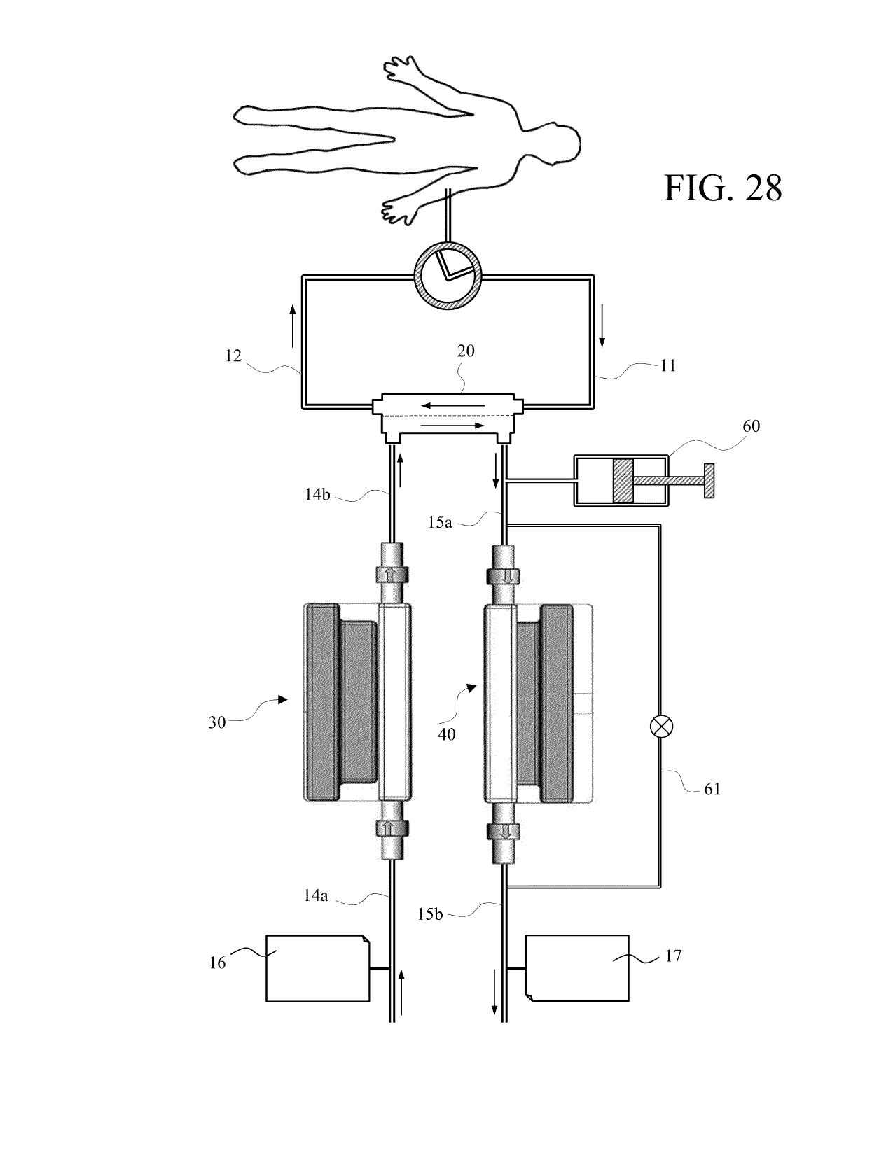

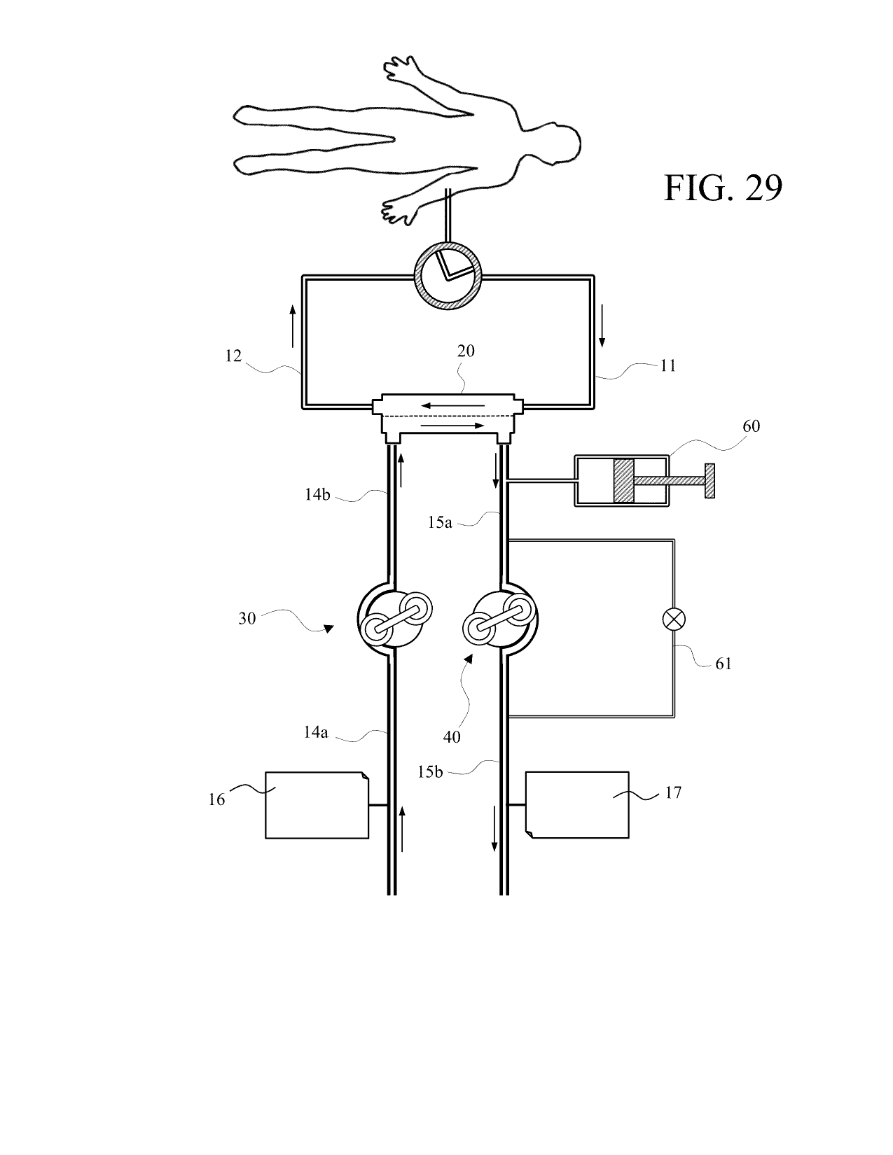

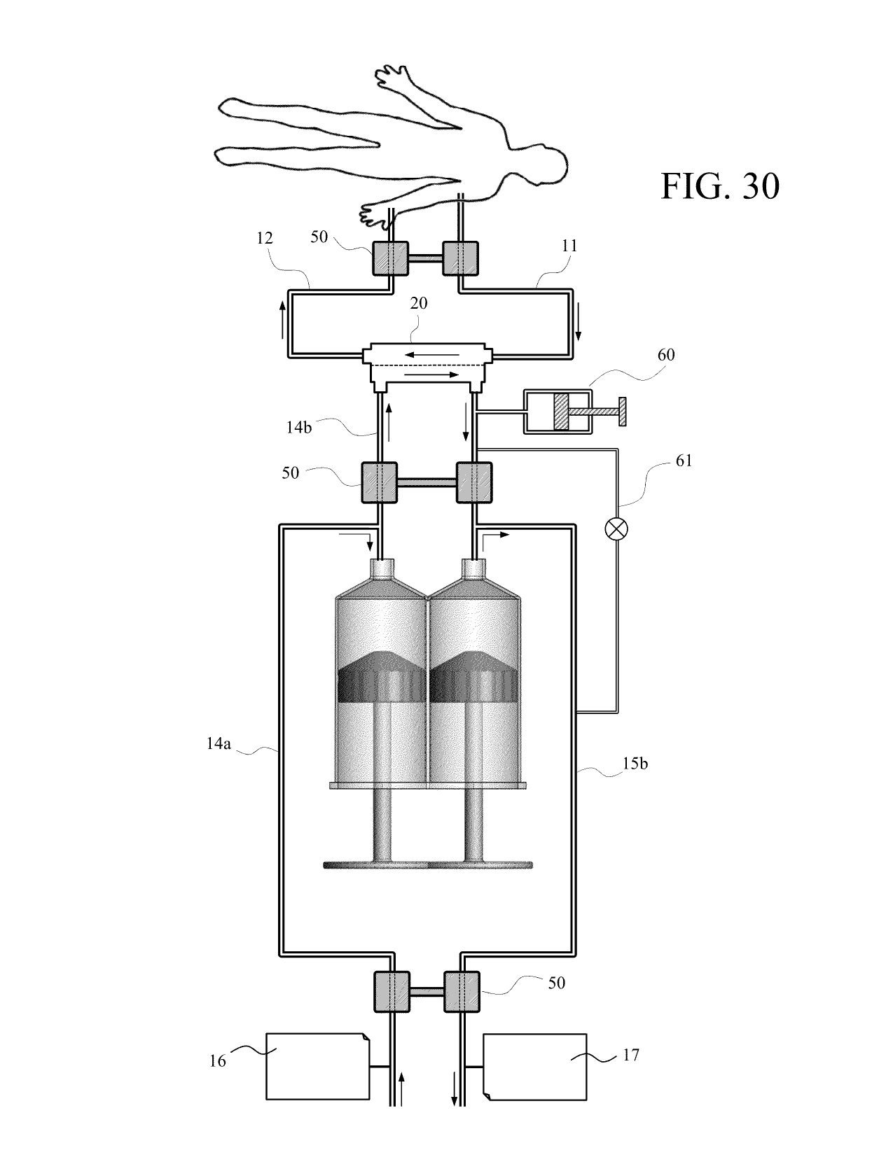

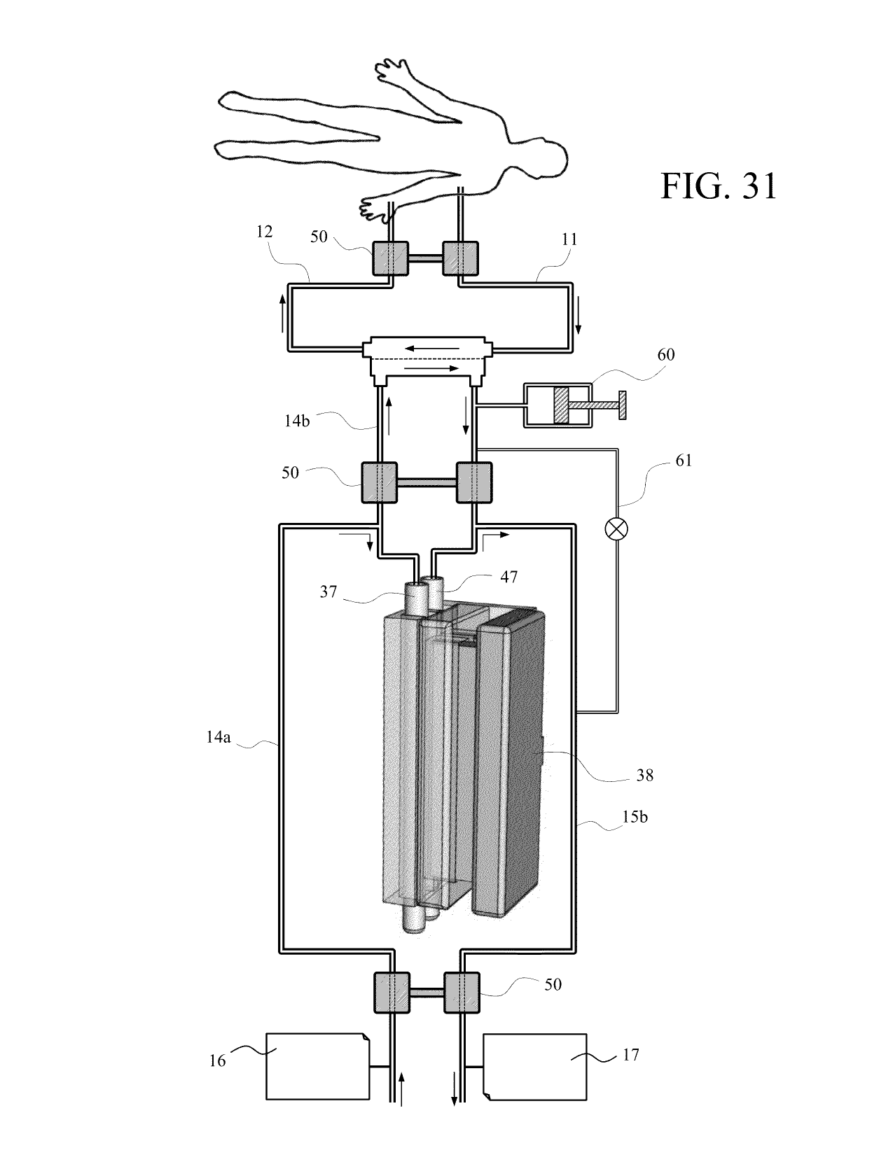

As stated above, the compression and expansion of the dialysate supply pump 30 and the dialysate discharge pump 40 configures a cycle of filtration and backfiltration. In the hemodialysis using the hemodialysis apparatus 10 according to embodiments of the present invention, the cycle of filtration and backfiltration is continuously repeated, removing water and waste products during the filtration and supplementing lost water during the backfiltration. That is, the dialysate pressure increases during the first phase, the supply phase or the compression phase, whereas it decreases during the second phase, the discharge phase, or the expansion phase. When the dialysate pressure fluctuates, the hemodialysis apparatus 10 according to embodiments of the present invention may further include a pressure-relief bypass 61 which connects between the first and second dialysate discharge tube 15a and 15b. FIGS. 28 to 31 illustrate the hemodialysis apparatus 10 having the pressure-relief bypass 61.

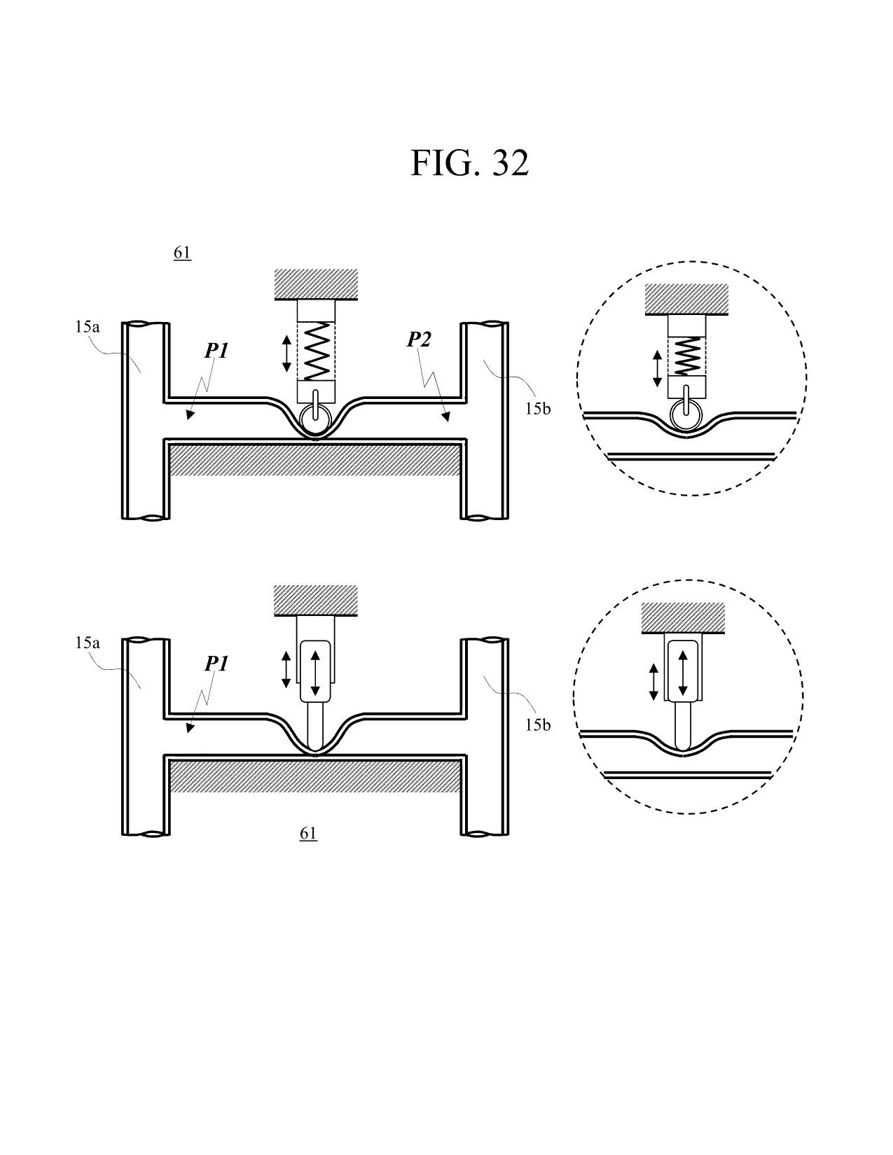

When the dialysate pressure of the hemodialyzer 20, which is equal to the pressure of the first dialysate discharge tube 15a, increases above the permissible range, dialysate of the hemodialyzer may be removed to the second dialysate discharge tube 15b through the pressure-relief bypass 61. On the contrary, when the dialysate pressure inside the hemodialyzer 20 decreases below the permissible range, dialysate may be supplemented to the hemodialyzer through the pressure-relief bypass 61 so as to compensate the pressure decrease inside the hemodialyzer 20. An exemplary pressure-relief bypass is illustrated in FIG. 32. Under the normal operation, the pressure-relief bypass remains closed due to the compression of the spring. However, the dialysate pressure of the hemodialyzer exceeds the spring compression, the spring moves upward in the drawing and the pressure-relief bypass opens. The pressure-relief bypass 61 is not limited to be opened or closed by the dialysate pressure of the hemodialyzer 20. Rather, the pressure-relief bypass can be opened or closed by the pressure of the second dialysate supply tube 14b, the pressure difference of the both tubes connected by the pressure-relief bypass 61, or the transmembrane pressure (TMP) of the hemodialyzer 20.

The pressure values that can open or close the pressure-relief bypass 61 may be dependent on the hemodialysis membrane 22 that is used. In general, the hemodialysis membrane has a limit of the pressure difference between blood and dialysate flowing therethrough to prevent the hemodialysis membrane from being damaged. In this instance, the pressure-relief bypass 61 is provided so that the dialysate pressure is lowered by the removal of dialysate downstream of the dialysate discharge pump 40, or the dialysate pressure can be increased by supplementing dialysate from the second dialysate discharge tube 15b. In other words, the pressure of dialysate flowing through the hemodialyzer can be maintained in a permissible range due to the operation of the pressure-relief bypass 61. Finally, the pressure-relief bypass may be modified to connect between the first and second dialysate supply tube 14a and 14b.

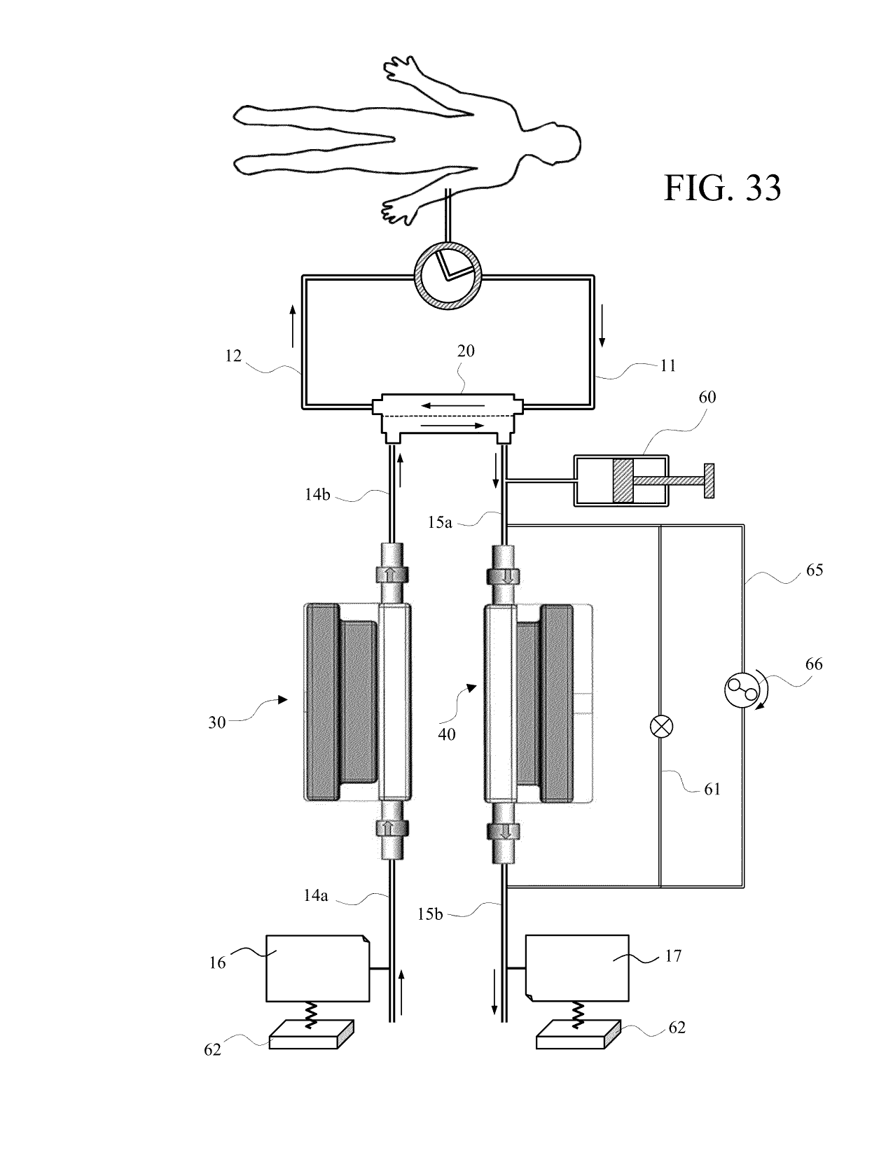

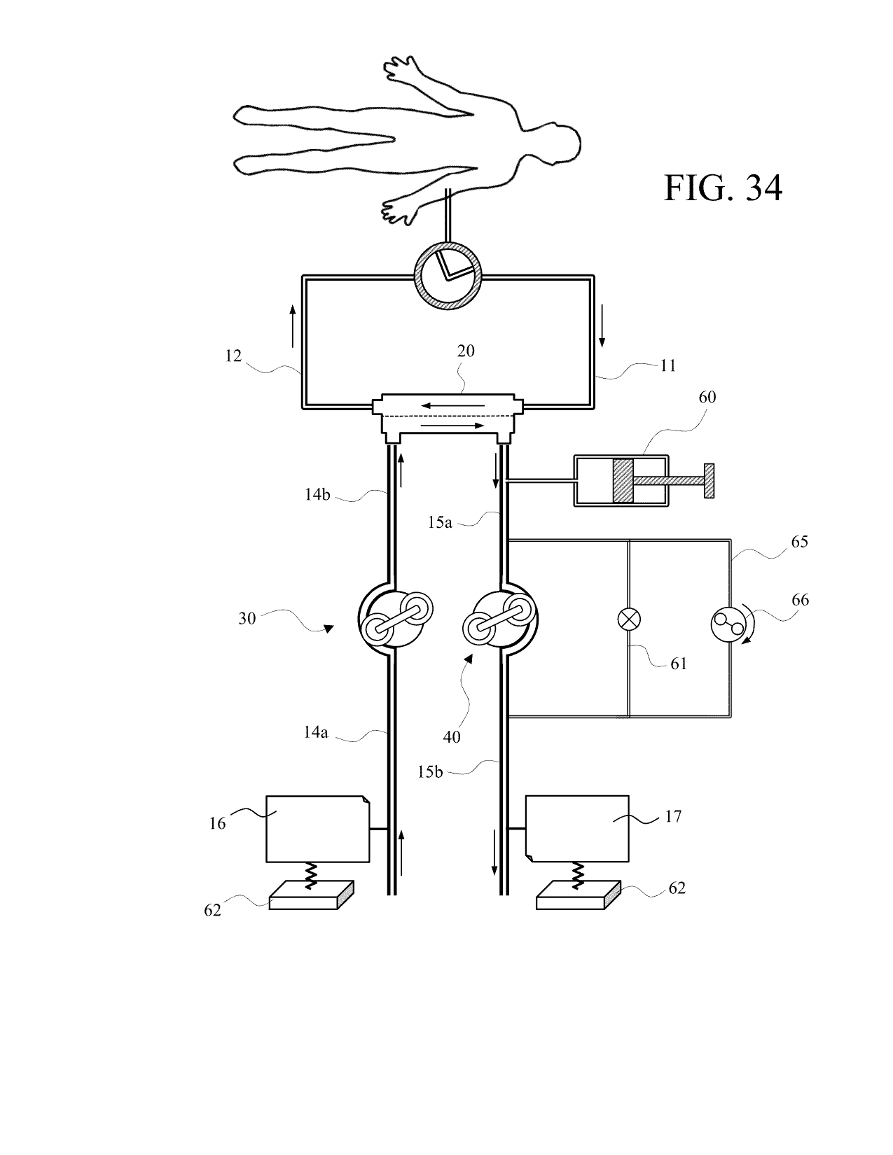

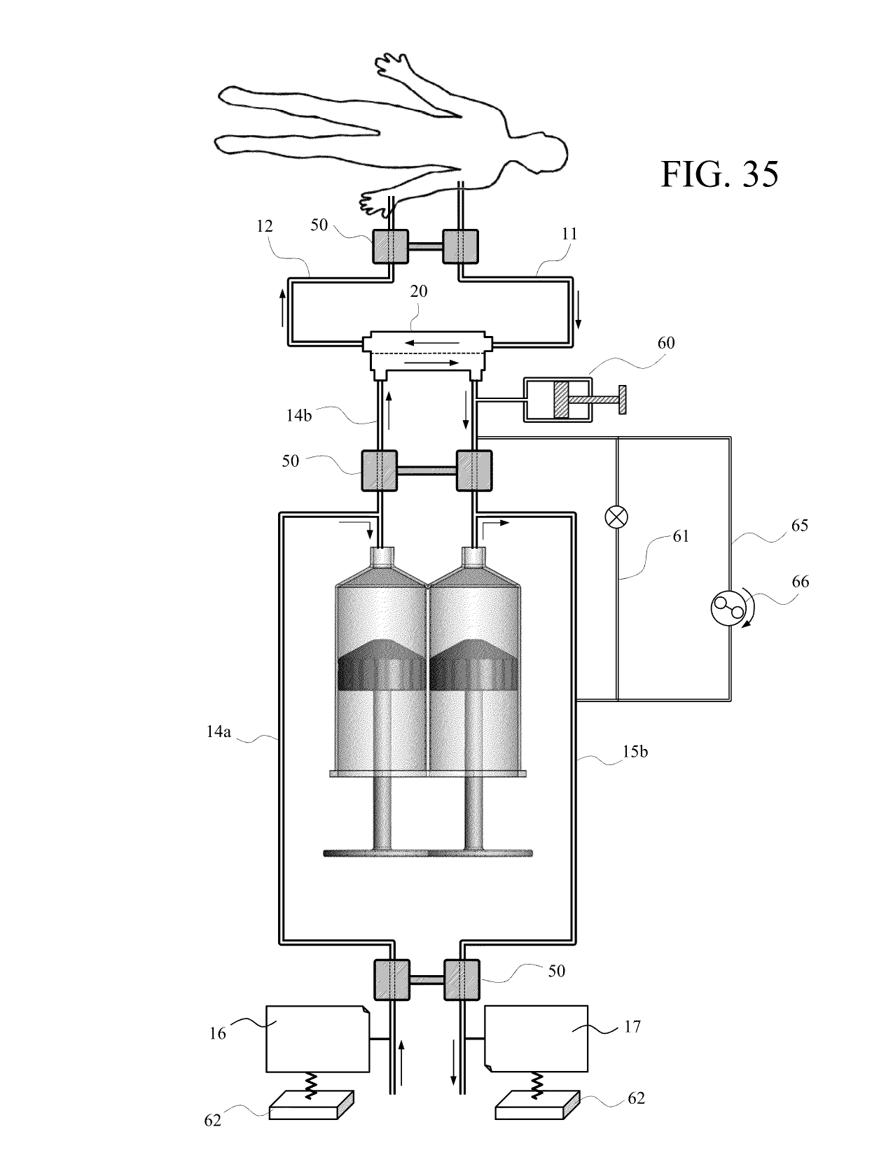

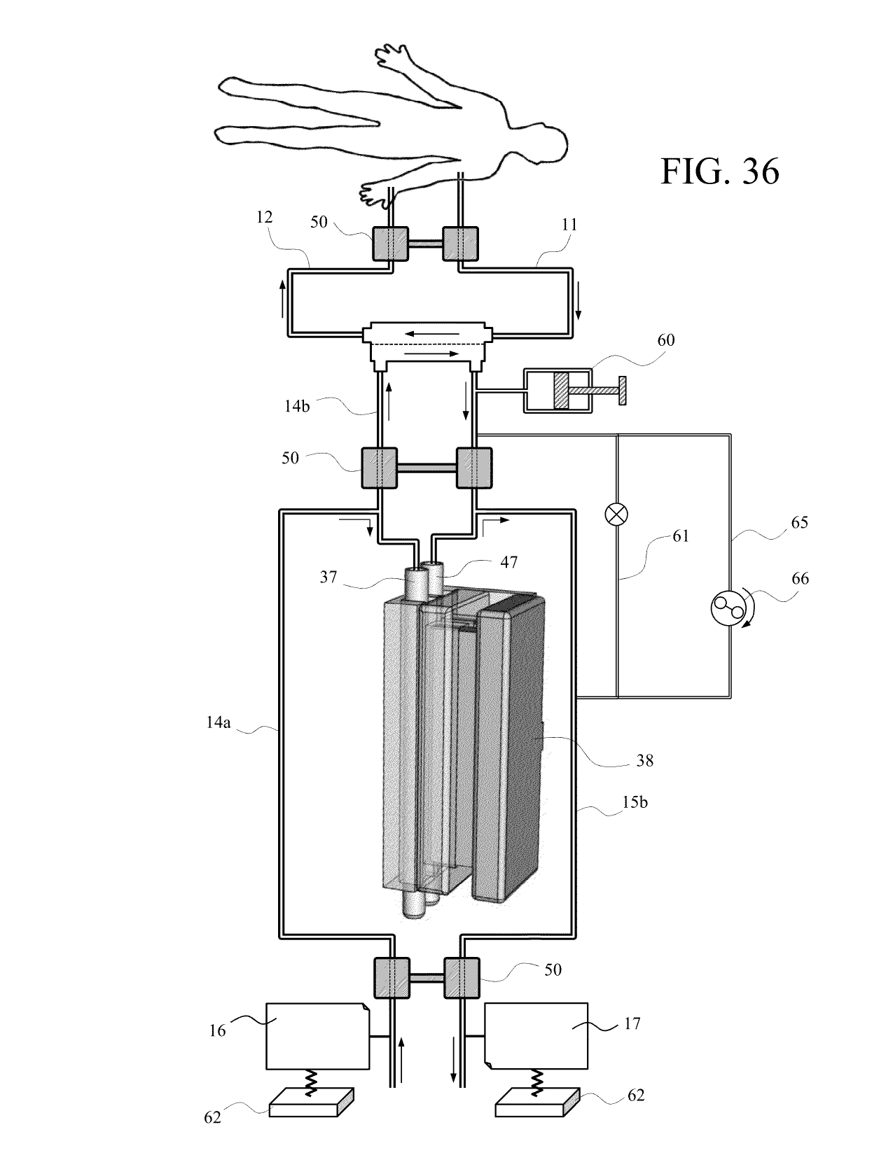

In addition, the hemodialysis apparatus 10 according to embodiments of the present invention may be further provided with a method to measure the amount of dialysate supplied to the hemodialyzer and the amount of dialysate collected from the hemodialyzer 20. For example, as shown in FIGS. 33 to 36, a balance may be provided to measure the amount of dialysate supplied from the dialysate supply tank 16 and then collected in the dialysate recovery tank 17. Since water is accumulated in the body of a patient with renal disease due to the absence of the kidney function, it is important to remove excess water from the body as well as remove waste product from the body upon hemodialysis. The amount of water that was removed from a patient during hemodialysis may be determined using the difference between the amount of dialysate that was supplied to and collected from the hemodialyzer. Alternatively, the amount of dialysate supplied and discharged can be measured by a flowmeter provided on the dialysate supply tube 14 and the dialysate discharge tube 15. The hemodialysis apparatus 10 according to embodiments of the present invention may have other methods to measure the amount of dialysate that is supplied to the hemodialyzer and the amount of dialysate that is collected from the hemodialyzer 20.

As described above, it is important to remove excess water from the body of a patient with renal disease. Thus, as shown in FIGS. 33 to 36, the hemodialysis apparatus 10 according to embodiments of the present invention may additionally have an auxiliary discharge tube 65 connecting between the first and second dialysate discharge tube 15a and 15b, and an auxiliary discharge pump 66 disposed on the auxiliary discharge tube to additionally pull dialysate of the hemodialyzer 20 toward the dialysate recovery tank 17. In a situation where the supply amount of dialysate by the dialysate supply pump 30 and the discharge amount of dialysate by the dialysate discharge pump 40 are equal to each other, when the auxiliary discharge pump 66 operates, water may be additionally discharged out of blood, thereby removing excess water accumulated in the body of a patient.

Finally, the hemodialysis apparatus 10 according to embodiments of the present invention may be modified into a configuration that includes a blood pump to control the blood flow rate through the blood tube 11 and 12. The blood pump can be disposed on the blood tube 11 and 12, and replace the blood flow controller 13 or the flow control device 50.

Thus, the hemodialysis apparatuses 10 according to the embodiments can improves the hemodialysis efficiency by allowing a pressure difference between blood and dialysate to be alternately changed into positive (+) and negative (−) values, and enables simplification and miniaturization of the hemodialysis apparatus and provides convenience in installation and use.

The above-disclosed subject matter is to be considered illustrative, and not restrictive, and the appended claims are intended to cover all such modifications, enhancements, and other embodiments, which fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A hemodialysis apparatus comprising:
a hemodialyzer in which a mass transfer occurs between blood and dialysate;
a first blood tube connecting between the hemodialyzer and a patient and allowing blood to flow therethrough;
a second blood tube connecting between the hemodialyzer and a patient and allowing blood to flow therethrough;
a blood flow controller controlling blood flow through the first and second blood tubes,
a dialysate supply pump supplying dialysate to the hemodialyzer; and
a dialysate discharge pump discharging dialysate out of the hemodialyzer.

2. The hemodialysis apparatus of claim 1, wherein:
the dialysate supply pump comprises a supply tube pressurizing member which pressurizes the dialysate supply tube, a supporting wall which supports the dialysate supply tube, and a supply tube one-way valve provided at both sides of the dialysate supply tube pressurized by the supply tube pressurizing member so as to prevent dialysate in the tube from flowing backward, and
the dialysate discharge pump comprises a discharge tube pressurizing member which pressurizes the dialysate discharge tube, a supporting wall which supports the dialysate discharge tube, and a discharge tube one-way valve provided at both sides of the dialysate discharge tube pressurized by the discharge tube pressurizing member to prevent dialysate in the tube from flowing backward.

3. The hemodialysis apparatus of claim 2, comprising:
a tube pressurizing member protrusion provided on the pressurizing surface of the tube pressurizing member which contacts with the dialysate tube; or
a supporting wall protrusion disposed on the supporting wall to block the dialysate flow through the dialysate tube when the dialysate tube is pressurized by the tube pressurizing member.

4. The hemodialysis apparatus of claim 3, further comprising a volume chamber to store dialysate, wherein the volume chamber is connected to the dialysate discharge tube connecting between the hemodialyzer and the dialysate discharge pump, or to the dialysate supply tube connecting between the hemodialyzer and the dialysate supply pump.

5. The hemodialysis apparatus of claim 4, wherein the blood flow controller comprises:
a housing having an internal space;
a flow port disposed on the housing;
a rotor disposed inside the housing to connect flow passage between the flow ports; and
a rotor driver for rotating the rotor.

6. The hemodialysis apparatus of claim 4, wherein the blood flow controller comprises:
a flow-blocking member pressurizing the blood tube;
a flow-blocking wall supporting the blood tube pressurized by the flow-blocking member; and
a flow-blocking member driver driving the flow-blocking member.

7. The hemodialysis apparatus of claim 4, wherein the blood flow controller comprises a blood tube one-way valve disposed on the blood tube, allowing blood flowing therethrough to flow in one direction.

8. The hemodialysis apparatus of claim 4, wherein the dialysate flow comprises:
a first phase where the supply tube pressurizing member pressurizes the dialysate supply tube to supply dialysate therein to the hemodialyzer, while the dialysate discharge tube is pressurized and blocked by the discharge tube pressurizing member;
a second phase where the discharge tube pressurizing member expands the dialysate discharge tube and dialysate of the hemodialyzer flows into the dialysate discharge tube, while the dialysate supply tube is pressurized and blocked by the supply tube pressurizing member; and
a third phase where the supply tube pressurizing member expands the dialysate supply tube, allowing dialysate to flow into the dialysate supply tube, and the discharge tube pressurizing member pressurizes the dialysate discharge tube, enabling the dialysate therein to be discharged.

9. The hemodialysis apparatus of claim 8, wherein:
the blood flow controller opens a blood flow passage through the second blood tube and blocks a blood flow passage through the first blood tube during the first phase, and
the blood flow controller opens a blood flow passage through the first blood tube and blocks a blood flow passage through the second blood tube during the second phase.

10. The hemodialysis apparatus of claim 4, further comprising:
a pressure-relief bypass connecting the first dialysate discharge tube and the second dialysate discharge tube, or connecting the first dialysate supply tube and the second dialysate supply tube.

11. The hemodialysis apparatus of claim 10, further comprising:
a balance to measure the amount of dialysate supplied to the hemodialyzer and the amount of dialysate collected from the hemodialyzer; or
a flowmeter disposed on the dialysate tube to measure the amount of dialysate supplied to the hemodialyzer and the amount of dialysate collected from the hemodialyzer.

12. The hemodialysis apparatus of claim 11, comprising:
an auxiliary discharge tube connecting the first dialysate discharge tube and the second dialysate discharge tube; and
an auxiliary discharge pump disposed on the auxiliary discharge tube to additionally remove dialysate from the hemodialyzer.

13. The hemodialysis apparatus of claim 1, wherein the dialysate supply pump and the dialysate discharge pump comprise:
a roller that transfers dialysate by squeezing the dialysate tube; and
a roller driver operating the roller, and
a dialysate flow passage through the dialysate tube is blocked when the roller squeezes the dialysate tube.

14. The hemodialysis apparatus of claim 13, wherein the dialysate flow comprises:
a supply phase in which a flow rate of dialysate that is supplied to the hemodialyzer by the dialysate supply pump is larger than a flow rate of dialysate that is discharged from the hemodialyzer by the dialysate discharge pump, and
a discharge phase in which a flow rate of dialysate that is supplied to the hemodialyzer by the dialysate supply pump is smaller than a flow rate of dialysate that is discharged from the hemodialyzer by the dialysate discharge pump.

15. The hemodialysis apparatus of claim 14, wherein:
the blood flow controller opens a blood flow passage through the second blood tube and blocks a blood flow passage through the first blood tube during the supply phase, and
the blood flow controller opens a blood flow passage through the first blood tube and blocks a blood flow passage through the second blood tube during the discharge phase.

16. The hemodialysis apparatus of claim 15, wherein the blood flow controller comprises:
a housing having an internal space;
a flow port disposed on the housing;
a rotor disposed inside the housing to connect flow passage between the flow ports; and
a rotor driver for rotating the rotor.

17. The hemodialysis apparatus of claim 15, wherein the blood flow controller comprises:
a flow-blocking member pressurizing the blood tube;
a flow-blocking wall supporting the blood tube pressurized by the flow-blocking member; and
a flow-blocking member driver driving the flow-blocking member.

18. The hemodialysis apparatus of claim 15, wherein the blood flow controller comprises a blood tube one-way valve disposed on the blood tube, allowing blood flowing therethrough to flow in one direction.

19. The hemodialysis apparatus of claim 15, further comprising:
a pressure-relief bypass connecting the first dialysate discharge tube and the second dialysate discharge tube, or connecting the first dialysate supply tube and the second dialysate supply tube.

20. The hemodialysis apparatus of claim 19, comprising:
an auxiliary discharge tube connecting the first discharge tube and the second discharge tube; and
an auxiliary discharge pump disposed on the auxiliary discharge tube to additionally remove dialysate from the hemodialyzer.
